SEQUENCE LISTING (4 sequences)

SEQ ID NO: 1 - DNA, 2277 bp, Artificial Sequence
  albumin-GCSF fusion
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccc

SEQ ID NO: 2 - protein, 759 aa, Artificial Sequence
  albumin-GCSF fusion
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 4 - protein, 609 aa, Homo sapiens [sequence omitted]

(19) United States
(12) Patent Application Publication
(10) Pub. No.: US 2014/0271538 A1
(43) Pub. Date: Sep. 18, 2014

(54) Recombinant Human Albumin-Human Granulocyte Colony Stimulating Factor for the Prevention of Neutropenia in Pediatric Patients

(71) Applicant: Teva Pharmaceutical Industries Ltd., Petach-Tikva (IL)

(72) Inventors: Anton BUCHNER, Senden (DE); Andreas Lammerich, Ilertissen (DE); Noa Avisar, Zoran (IL)

(73) Assignee: Teva Pharmaceutical Industries Ltd., Petach-Tikva (IL)

(21) Appl. No.: 14/213,989

(22) Filed: Mar. 14, 2014

Related U.S. Application Data
(60) Provisional application No. 61/789,050, filed on Mar. 15, 2013.

(51) Int. Cl. C07K 14/53; C07K 14/765

(52) U.S. Cl. CPC C07K 14/53; C07K 14/765
USPC 424/85.1

(57) ABSTRACT

Disclosed are methods and compositions for treating, preventing and ameliorating conditions and diseases characterized by a lowered white blood cell count, including neutropenia, in human patients that are less than 18 years old. The methods and compositions described herein include a fusion polypeptide comprising human serum albumin protein (“HSA”) and human granulocyte-colony stimulating factor (“G-CSF”).

### REFERENCE TO RELATED APPLICATIONS

The disclosure of related application U.S. 61/789,050, filed Mar. 15, 2013, is herein incorporated by reference in its entirety.

### REFERENCE TO A SEQUENCE LISTING SUBMITTED ELECTRONICALLY VIA EFS-WEB

The content of the electronically submitted sequence listing (Name: 2873_{—}1290001_SEQLISTING.ascii, Size: 17,250 bytes; and Date of Creation: Mar. 10, 2014) filed with the application is herein incorporated by reference in its entirety.

### BACKGROUND

Leukopenia is a reduction in the circulating White Blood Cells (WBC) and is often defined as a WBC count less than 4000/mL. The main cells involved in leukopenia are neutrophils. However a reduced number of lymphocytes, monocytes, eosinophils, or basophils may also contribute to the decreased total white blood cell count (Merck Manual, 17th edition).

Neutropenia is characterized by a reduction in the blood neutrophil count, often leading to increased susceptibility to bacterial and fungal infections. Neutropenia is classified by the neutrophil count and the relative risk of infection: mild (1000 to 1500/mL), moderate (grade 3, 500 to 1000/mL), or severe (grade 4, <500/mL). Acute and severe neutropenia is a life-threatening condition as it predisposes the patient to rapidly fatal infections (Merck Manual, 17th edition).

Neutropenia can be caused by impaired production of neutrophils in the bone marrow, accelerated destruction of neutrophils, or sequestration of neutrophils. Acute neutropenia may occur over a few days when neutrophil use is rapid and production is severely impaired. Chronic neutropenia may last for many months and is often caused by reduced production or sequestration of neutrophils in the spleen. Neutropenia may be classified by whether it arises secondary to factors extrinsic to marrow myeloid cells or whether an intrinsic defect appears to be present in the myeloid progenitors (Merck Manual, 17th edition).

Neutropenia and its infectious complications are among the most common and serious adverse effects of cytotoxic chemotherapy and other cancer therapies such as radiation therapy, biotherapy, targeted therapy and bone marrow transplantation. Cytotoxic chemotherapy, which works by destroying fast-growing cells, induces neutropenia because of the high proliferative rate of neutrophil precursors and the rapid turnover of blood neutrophils (Merck Manual, 17th edition). The most common symptoms of neutropenia in patients undergoing chemotherapy include fever, mouth sores, and ear infections. Patients with profound neutropenia often suffer from pyogenic infections such as septicemia, cutaneous cellulitis, liver abscesses, furunculosis, pneumonia, stomatitis, gingivitis, perirectal inflammation, colitis, sinusitis, and otitis media. Chemotherapy may have to be delayed until the body can produce more neutrophils, and a lower dosage may have to be given, resulting in the treatment being less effective.

The prevention of neutropenia is an important objective of daily oncological practice, both for patient safety as neutropenic patients are vulnerable to life threatening infections, and cost efficiency, as hospitalization and intensive care treatments of these infections are costly.

The risk and severity of febrile neutropenia in adults and pediatrics vary depending on chemotherapy drug regimens as well as patient characteristics and factors such as age or comorbid conditions. Chemotherapy treatment in pediatric cancer care is often characterized by high intensive dosing. The infection rate of septicemia and invasive mycoses in pediatrics with solid tumors is associated with the intensity of the chemotherapy and is greater for intensive treatments compared with less intensive treatments. Due to their high recovery and resilience, pediatric patients are often exposed to a more myelosuppressive chemotherapy treatment (e.g., intense dose or dense dose chemotherapy regimen) compared to adults.

An analysis of 4 studies of the European Organization for Research and Treatment of Cancer (EORTC) comparing the outcome of febrile neutropenic episodes in pediatrics versus adults presented the following results: pediatric patients had less frequently a defined site of infection than adults and where they had a defined site of infection they experienced more upper respiratory tract but fewer lung infections. There was a similar low incidence of shock at presentation in the two groups but the pediatric patients' median neutrophil count was lower, and their median duration of granulocytopenia before the trial was shorter. The incidence of bacteremia was similar between adults and pediatric patients. However, clinically documented infection was less frequent in pediatrics, and fever of unknown origin was more frequent in pediatrics. Compared with adults, pediatric patients developed more streptococcal bacteremias and fewer staphylococcal bacteremias, although the relative incidence of various gram-negative species was similar in adults and pediatric patients. In general, the pediatric patients had a better overall success rate and lower mortality than adults. Death from infection was only 1% in pediatrics versus 4% in adults and time to body temperature normalization following fever was shorter in pediatrics. In the younger age group, high temperature, prolonged neutropenia before the trial and septic shock were prognostic indicators for the presence of bacteremia. Solid tumor patients were significantly less likely to have a bacteremia.

Currently, there are several recombinant human G-CSF products available as supportive treatment during chemotherapy administration. In Europe and the USA the daily administration of short acting filgrastim (Neupogen®, Amgen, Thousand Oaks, Calif., USA) is approved for the treatment of chemotherapy-induced neutropenia and febrile neutropenia in adults and in the pediatric population.

Filgrastim has been used in pediatric patients, particularly in the treatment of chemotherapy-induced neutropenia and severe chronic neutropenia. Filgrastim can be administered to decrease the incidence of infection, as manifested by febrile neutropenia, in patients with nonmyeloid malignancies receiving myelosuppressive anti-cancer drugs associated with a significant incidence of severe neutropenia with fever.

A second generation, pegylated G-CSF product, pegfilgrastim, which has a slower clearance, allowed for a G-CSF treatment at a frequency of once per chemotherapy cycle. The efficacy of this product is comparable to daily filgrastim therapy (Neulasta® SmPC). There are no recommendations for the use of pegfilgrastim in pediatric patients.

To date, the need for an efficious and safe, long acting G-CSF, that does not require daily administration during chemotherapy treatment cycles, for the treatment of Neutropenia in pediatric patients, still remains.

### SUMMARY

Described herein are methods and compositions useful for the treatment, amelioration and prevention of conditions characterized by a lower than normal white blood cell count in a pediatric population. Such conditions include but are not limited to leukopenia and neutropenia.

In a first embodiment, described is a method of treating or preventing neutropenia in a human subject comprising administering to a human subject exhibiting neutropenia or at risk of developing neutropenia, recombinant human albumin-human granulocyte colony stimulating factor in an amount effective to treat the subject wherein the subject is less than 18 years old. In other embodiments of the invention, the human subject is less than two years old, one month to 18 years old, 2-17 years old, 2-11 years old, or 12-17 years old. In an exemplary embodiment, the human subject can be suffering from a non-myeloid malignancy and receiving at least one myelosuppressive anti-cancer drug associated with a clinically significant incidence of febrile neutropenia.

In a second embodiment, described is a method of decreasing the incidence of infection, as manifested by febrile neutropenia, in a human subject with non-myeloid malignancies and receiving at least one myelosuppressive anti-cancer drug associated with a clinically significant incidence of febrile neutropenia, comprising administering to the subject recombinant human albumin-human granulocyte colony stimulating factor in an amount effective to treat the subject wherein the subject is less than 18 years old. In other embodiments of the invention the human subject is less than two years old, one month to 18 years old, 2-17 years old, 2-11 years old, or 12-17 years old.

In some embodiments, the human subject is male. In some embodiments, the human subject is a female.

In some embodiments, the human subject weighs not more than 29.8 Kg. In some embodiments, the human subject weighs at least 15 Kg. In some embodiments, the human subject weighs at least 12.5 Kg. In some embodiments, the human subject weighs at least 10 Kg. In some embodiments, the human subject weights about 10 to about 30 Kg.

In some embodiments, the human subject is suffering from a non-myeloid malignancy and is receiving at least one myelosuppressive anti-cancer drug associated with a clinically significant incidence of neutropenia. In some embodiments, the anti-cancer drug is associated with a clinically significant incidence of febrile neutropenia.

In some embodiments, the human subject has a non-myeloid malignancy that is a solid tumor or lymphoma. In some embodiments, the human subject has a non-myeloid malignancy that is a solid tumor.

In some embodiments, after administration of human albumin-human granulocyte colony stimulating factor grade 4 neutropenia in the subject is eliminated, grade 4 neutropenia in the subject is reduced, the duration of severe neutropenia is reduced in the subject, the incidence of severe neutropenia is reduced in the subject, the frequency of febrile neutropenia is reduced in the subject, the duration of grade 4 neutropenia in the subject is less than 5 days, the duration of grade 3 neutropenia in the subject is eliminated, the duration of grade 3 neutropenia in the subject is decreased, or any combination thereof.

In some embodiments, administering recombinant human albumin-human granulocyte colony stimulating factor induces a rise in white blood cells (WBC).

In some embodiments, after administration of human albumin-human granulocyte colony stimulating factor the number of neutrophils is increased in the subject, a decrease in the number of neutrophils is inhibited in the subject, the nadir absolute neutrophil count (ANC) is increased in the subject, the recovery ANC is increased in the subject, the time to ANC recovery is reduced in the subject, or any combination thereof.

In some embodiments, the amount of recombinant human albumin-human granulocyte colony stimulating factor administered to the subject is selected from the group consisting of: from about 200 μg/kg to about 700 μg/kg; from about 300 μg/kg to about 670 μg/kg; about 300 μg/kg; about 670 μg/kg; from about 0.4 mg to about 50 mg; less than 50 mg; less than 40 mg; less than 30 mg; less than 20 mg; less than 10 mg; and less than 5 mg.

In some embodiments, the neutropenia is selected from the group consisting of primary neutropenia, acute neutropenia, severe chronic neutropenia (SCN), severe congenital neutropenia (Kostmann's syndrome), severe infantile genetic agranulocytosis, benign neutropenia, cyclic neutropenia, chronic idiopathic neutropenia, secondary neutropenia, syndrome associated neutropenia, febrile neutropenia and immune-mediated neutropenia.

In some embodiments, neutropenia is caused or associated with radiation, alcoholism, drugs, an allergic disorder, aplastic anemia, autoimmune disease, T-γ lymphoproliferative disease (T-γ LPD), myelodysplasia, myelofibrosis, dysgammaglobulinemia, paroxysmal nocturnal hemoglobinuria, cancer, vitamin B12 deficiency, folate deficiency, viral infection, bacterial infection, spleen disorder, hemodialysis, transplantation, leukemia, myeloma, lymphoma, a metastatic solid tumor which infiltrates and replaces the bone marrow, a toxin, bone marrow failure, Schwachman-Diamond syndrome, cartilage-hair hypoplasia, dyskeratosis congenita, glycogen storage disease type IB, splenomegaly of any cause, or an intrinsic defect in myeloid cells or their precursors.

In some embodiments, recombinant human albumin-human granulocyte colony stimulating factor is administered at a time selected from the group consisting of: at least 18 hours after administration of the myelosuppressive anti-cancer drug; at least 21 hours after administration of the myelosuppressive anti-cancer drug; at least 24 hours after administration of the myelosuppressive anti-cancer drug; and at least 27 hours after administration of the myelosuppressive anti-cancer drug. In some embodiments, recombinant human albumin-human granulocyte colony stimulating factor is administered prior to administration of the myelosuppressive anti-cancer drug.

In some embodiments, administering recombinant human albumin-human granulocyte colony stimulating factor prior to the myelosuppressive anti-cancer drug induces a rise in WBC and/or induces a rise in ANC.

In some embodiments, the myelosuppressive anti-cancer drug comprises Vincristine, Doxorubicin, Cyclophosphamide, Ifosfamide and Etoposide (VDC/IE) or Vincristine, Ifosfamide, Doxorubicin, and Etoposide (VIDE) chemotherapy regime.

In some embodiments, the VIDE chemotherapy regime comprises about 1.5 mg/m² Vincristine, about 3.0 g/m² Ifosfamide, about 20 mg/m² Doxorubicin, and about 150 mg/m² Etoposide for at least one treatment cycle. In some embodiments, about 1.5 mg/m² Vincristine is given on Day 1, about 3.0 g/m² Ifosfamide is given on Day 1, Day 2, and Day 3, about 20 mg/m² Doxorubicin is given on Day 1, Day 2, and Day 3, and about 150 mg/m² Etoposide is given on Day 1, Day 2, and Day 3 of the treatment cycle for at least one treatment cycle.

In some embodiments, the VDC/IE chemotherapy regime comprises about 2.0 mg/m² Vincristine, about 75 mg/m² Doxorubicin, about 1200 mg/m² Cyclophosphamide, about 1800 mg/m²/day Ifosfamide, and about 100 mg/m²/day Etoposide for at least one treatment cycle. In some embodiments, about 2.0 mg/m² Vincristine is given on Day 1, Day 8, and Day 15, about 75 mg/m² Doxorubicin is given on Day 1, about 1200 mg/m² Cyclophosphamide is given on Day 1 and Day 2 of a treatment cycle, and about 1800 mg/m²/day Ifosfamide and about 100 mg/m²/day Etoposide are given for 5 days of a treatment cycle for at least one treatment cycle. In some embodiments, about 2.0 mg/m² Vincristine is given on Day 1, Day 8, and Day 15, about 75 mg/m² Doxorubicin is given on Day 1, about 1200 mg/m² Cyclophosphamide is given on Day 1 and Day 2 (e.g., by intravenous infusion) on chemotherapy treatment cycles 1 and 3, and about 1800 mg/m²/day Ifosfamide and about 100 mg/m²/day Etoposide are given for 5 days on treatment cycles 2 and 4.

In some embodiments, human albumin-human granulocyte colony stimulating factor is administered at a time selected from the group consisting of: at least 18 hours after administration of Ifosfamide, Doxorubicin or Etoposide; at least 21 hours after administration of Ifosfamide, Doxorubicin or Etoposide; at least 24 hours after administration of Ifosfamide, Doxorubicin or Etoposide; and at least 27 hours after administration of Ifosfamide, Doxorubicin or Etoposide. In some embodiments, recombinant human albumin-human granulocyte colony stimulating factor is administered prior to administration of Ifosfamide, Doxorubicin or Etoposide.

In some embodiments, recombinant human albumin-human granulocyte colony stimulating factor is administered at a time selected from the group consisting of: at least 18 hours after administration of Cyclophosphamide; at least 21 hours after administration of Cyclophosphamide; at least 24 hours after administration of Cyclophosphamide; and at least 27 hours after administration of Cyclophosphamide. In some embodiments, recombinant human albumin-human granulocyte colony stimulating factor is administered prior to administration of Cyclophosphamide.

In some embodiments, the myelosuppressive anti-cancer drug comprises a VDC/IE chemotherapy regime. In some embodiments, the myelosuppressive anti-cancer drug comprises a VIDE chemotherapy regime.

In some embodiments, the myelosuppresive anti-cancer drug comprises an Ifosfamide, Vincristine and Actinomycin D (IVA) chemotherapy regime. In some embodiments, the IVA chemotherapy regime comprises about 3.0 g/m² Ifosfamide, about 1.5 m g/m² Vincristine, and about 1.5 mg/m² Actinomycin D for at least one treatment cycle. In some embodiments, about 3.0 g/m² Ifosfamide is given on Day 1 and Day 2, about 1.5 mg/m² Vincristine is given on Day 1, Day 8, and Day 15, and 1.5 mg/m² Actinomycin D is given on Day 1 of at least one treatment cycle. In some embodiments, about 3.0 g/m² Ifosfamide is given on Day 1 and Day 2, about 1.5 mg/m² Vincristine is given on Day 1, Day 8, and Day 15, and about 1.5 mg/m² Actinomycin D is given on Day 1 of at least treatment Cycles 1, 2, and 4, and about 3.0 g/m² Ifosfamide is given on Day 1 and Day 2, about 1.5 mg/m² Vincristine is given on Day 1, and about 1.5 mg/m² Actinomycin D is given on Day 1 of at least treatment Cycle 3. In some embodiments, the recombinant human albumin-human granulocyte colony stimulating factor is administered at a time selected from the group consisting of: at least 18 hours after administration of Ifosfamide; at least 21 hours after administration of Ifosfamide; at least 24 hours after administration of Ifosfamide; and at least 27 hours after administration of Ifosfamide.

In some embodiments, the myelosuppresive anti-cancer drug comprises an Ifosfamide, Vincristine, Actinomycin D, and Doxorubicin (IVAd) chemotherapy regime. In some embodiments, the IVAd chemotherapy regime comprises about 3.0 g/m² Ifosfamide, about 1.5 mg/m² Vincristine, about 1.5 mg/m² Actinomycin D, and about 20 mg/m² Doxorubicin. In some embodiments, about 3.0 g/m² Ifosfamide is given on Day 1 and Day 2, about 1.5 mg/m² Vincristine is given on Day 1, Day 8, and Day 15, and about 20 mg/m² Doxorubicin is given twice daily on Day 1 and Day 2 of at least one treatment cycle; and about 3.0 g/m² Ifosfamide is given on Day 1 and Day 2, about 1.5 mg/m² Vincristine is given on Day 1, Day 8, and Day 15, and about 1.5 mg/m² Actinomycin D is given on Day 1 of at least one treatment cycle. In some embodiments, about 3.0 g/m² Ifosfamide is given on Day 1 and Day 2, about 1.5 mg/m² Vincristine is given on Day 1, Day 8, and Day 15, and about 20 mg/m² Doxorubicin is given twice daily on Day 1 and Day 2 of at lease treatment Cycles 1 and 4; about 3.0 g/m² Ifosfamide is given on Day 1 and Day 2, about 1.5 mg/m² Vincristine is given on Day 1, Day 8, and Day 15, and about 1.5 mg/m² Actinomycin D is given on Day 1 of at least treatment Cycle 2; and about 3.0 g/m² Ifosfamide is given on Day 1 and Day 2, about 1.5 mg/m² Vincristine is given on Day 1 and about 20 mg/m² Doxorubicin is given twice daily on Day 1 and Day 2 of at least treatment Cycle 3. In some embodiments, the recombinant human albumin-human granulocyte colony stimulating factor is administered at a time selected from the group consisting of: at least 18 hours after administration of Ifosfamide or Doxorubicin; at least 21 hours after administration of Ifosfamide or Doxorubicin; at least 24 hours after administration of Ifosfamide or Doxorubicin; and at least 27 hours after administration of Ifosfamide or Doxorubicin.

In some embodiments, ANC and/or WBC return to normal at a time period selected from the group consisting of: by day 4 after administration of the anti-cancer drug; by day 5 after administration of the anti-cancer drug; by day 6 after administration of the anti-cancer drug; by day 7 after administration of the anti-cancer drug; by day 8 after administration of the anti-cancer drug; by day 9 after administration of the anti-cancer drug; by day 10 after administration of the anti-cancer drug; by day 11 after administration of the anti-cancer drug; by day 12 after administration of the anti-cancer drug; by day 13 after administration of the anti-cancer drug; by day 14 after administration of the anti-cancer drug; and by day 15 after administration of the anti-cancer drug.

In some embodiments, administering recombinant human albumin-human granulocyte colony stimulating factor induces a rise in lymphocytes, monocytes, eosinophils, basophils, or any combination thereof.

In some embodiments, after administering recombinant human albumin-human granulocyte colony stimulating factor a decrease in the number of lymphocytes, monocytes, eosinophils, or basophils is inhibited in the subject.

In some embodiments, the recombinant human albumin-human granulocyte colony stimulating factor is administered in a composition comprising sodium phosphate, mannitol, trehalose dihydrate, polysorbate 80, or a combination thereof.

In some embodiments, the recombinant human albumin-human granulocyte colony stimulating factor is administered in a composition comprising 20 mM sodium phosphate, 180 mM mannitol, 60 mM trehalose dihydrate, 0.06% (w/v) polysorbate 80 or a combination thereof.

In some embodiments, the recombinant human albumin-human granulocyte colony stimulating factor composition is at a pH of 5.8-6.2. In some embodiments, the composition is at a pH of about 6.0.

In some embodiments, the recombinant human albumin-human granulocyte stimulating factor is administered parenterally. In some embodiments, the recombinant human albumin-human granulocyte stimulating factor is administered by subcutaneous injection.

In some embodiments, the recombinant human albumin-human granulocyte colony stimulating factor composition is in the form of a lyophilized cake prior to administration.

In some embodiments, the recombinant human albumin-human granulocyte colony stimulating factor is administered once per anti-cancer drug cycle.

In some embodiments, administration of recombinant human albumin-human granulocyte colony stimulating factor improves survival rates and/or decreases tumor progression.

In some embodiments, the human subject is receiving a chemotherapeutic agent, and the recombinant human albumin-human granulocyte stimulating factor is administered no more than once per chemotherapeutic cycle.

In some embodiments, the administration of recombinant human albumin-human granulocyte stimulating factor produces an AUC of about 1,000 to about 400,000 ng/mL*hr.

In some embodiments, the administration of recombinant human albumin-human granulocyte stimulating factor produces a Cmax of about 25 to about 2000 ng/mL.

In some embodiments, the elimination half-life of recombinant human albumin-human granulocyte stimulating factor is about 10 to about 75 hours.

In one embodiment (E1), the invention provides a method of treating or preventing neutropenia in a human subject comprising administering to a human subject exhibiting neutropenia or at risk of developing neutropenia, recombinant human albumin-human granulocyte colony stimulating factor in an amount effective to treat the subject and wherein the human subject is less than 18 years old.

In another embodiment (E2), the invention provides a method of E1 wherein the human subject is less than two years old. In another embodiment (E3), the invention provides the method of E1 wherein the human subject is one month to 18 years old. In another embodiment (E4), the invention provides the method of E1, wherein the human subject is 2-17 years old. In another embodiment (E5), the invention provides the method of E1, wherein the human subject is 2-11 years old. In another embodiment (E6), the invention provides the method of E1, wherein the human subject is 12-17 years old.

In another embodiment (E7), the invention provides the method of any one of E1 to E6, wherein the human subject is a male. In another embodiment (E8), the invention provides the method of any one of E1 to E6, wherein the human subject is a female.

In another embodiment (E9), the invention provides the method of any one of E1 to E8, wherein the human subject weighs not more than 29.8 Kg.

In another embodiment (E10), the invention provides the method of any one of E1 to E9, wherein the human subject is suffering from a non-myeloid malignancy and is receiving at least one myelosuppressive anti-cancer drug associated with a clinically significant incidence of neutropenia. In another embodiment (E11), the invention provides the method of E10, wherein the anti-cancer drug is associated with a clinically significant incidence of febrile neutropenia.

In another embodiment (E12), the invention provides a method of decreasing the incidence of infection, as manifested by febrile neutropenia, in a human subject with a non-myeloid malignancy and receiving at least one myelosuppressive anti-cancer drug associated with a clinically significant incidence of febrile neutropenia, comprising administering to the subject recombinant human albumin-human granulocyte colony stimulating factor in an amount effective to treat the subject and wherein the human subject is less than 18 years old.

In another embodiment (E13), the invention provides the method of E12, wherein the human subject is less than two years old. In another embodiment (E14), the invention provides the method of E12, wherein the human subject is one month to 18 years old. In another embodiment (E15), the invention provides the method of E12, wherein the human subject is 2-17 years old. In another embodiment (E16), the invention provides the method of E12, wherein the human subject is 2-11 years old. In another embodiment (E17), the invention provides the method of E12, wherein the human subject is 12-17 years old.

In another embodiment (E18), the invention provides the method of any one of E 12 to E17, wherein the human subject is a male. In another embodiment (E19), the invention provides the method of any one of E12 to E17, wherein the human subject is a female.

In another embodiment (E20), the invention provides the method of any one of E12 to E19, wherein the human subject weighs not more than 29.8 Kg.

In another embodiment (E21), the invention provides the method of any one of E10 to E20, wherein the non-myeloid malignancy is a solid tumor.

In another embodiment (E22), the invention provides the method of any one of E1 to E21, wherein (a) grade 4 neutropenia in the subject is eliminated; (b) grade 4 neutropenia in the subject is reduced; (c) the duration of severe neutropenia is reduced in the subject; (d) the incidence of severe neutropenia is reduced in the subject; (e) the frequency of febrile neutropenia is reduced in the subject; (f) the duration of grade 4 neutropenia in the subject is less than 5 days; (g) the duration of grade 3 neutropenia in the subject is eliminated; (h) the duration of grade 3 neutropenia in the subject is decreased; or (i) any combination thereof.

In another embodiment (E23), the invention provides the method of any one of E1 to E22, wherein administering recombinant human albumin-human granulocyte colony stimulating factor induces a rise in white blood cells (WBC).

In another embodiment (E24), the invention provides the method of any one of E1 to E23, wherein (a) the number of neutrophils is increased in the subject; (b) a decrease in the number of neutrophils is inhibited in the subject; (c) the nadir absolute neutrophil count (ANC) is increased in the subject; (d) the recovery ANC is increased in the subject; (e) the time to ANC recovery is reduced in the subject; or (f) any combination thereof.

In another embodiment (E25), the invention provides the method of any one of E1 to E24, wherein the amount of recombinant human albumin-human granulocyte colony stimulating factor administered to the subject is selected from the group consisting of: (a) from about 200 μg/kg to about 700 μg/kg; (b) from about 300 μg/kg to about 670 μg/kg; (c) about 300 μg/kg; (d) about 670 μg/kg; (e) from about 0.4 mg to about 50 mg; (f) less than 50 mg; (g) less than 40 mg; (h) less than 30 mg; (i) less than 20 mg; (j) less than 10 mg; and (k) less than 5 mg.

In another embodiment (E26), the invention provides the method of any one of E1 to E25, wherein the neutropenia is selected from the group consisting of primary neutropenia, acute neutropenia, severe chronic neutropenia (SCN), severe congenital neutropenia (Kostmann's syndrome), severe infantile genetic agranulocytosis, benign neutropenia, cyclic neutropenia, chronic idiopathic neutropenia, secondary neutropenia, syndrome associated neutropenia, febrile neutropenia and immune-mediated neutropenia.

In another embodiment (E27), the invention provides the method of any one of E1 to E26, wherein neutropenia is caused or associated with radiation, alcoholism, drugs, an allergic disorder, aplastic anemia, autoimmune disease, T-γ lymphoproliferative disease (T-γ LPD), myelodysplasia, myelofibrosis, dysgammaglobulinemia, paroxysmal nocturnal hemoglobinuria, cancer, vitamin B12 deficiency, folate deficiency, viral infection, bacterial infection, spleen disorder, hemodialysis, transplantation, leukemia, myeloma, a metastatic solid tumor which infiltrates and replaces the bone marrow, a toxin, bone marrow failure, Schwachman-Diamond syndrome, cartilage-hair hypoplasia, dyskeratosis congenita, glycogen storage disease type IB, splenomegaly of any cause, or an intrinsic defect in myeloid cells or their precursors.

In another embodiment (E28), the invention provides the method of any one of E10 to E27, wherein recombinant human albumin-human granulocyte colony stimulating factor is administered at a time selected from the group consisting of: (a) at least 18 hours after administration of the myelosuppressive anti-cancer drug; (b) at least 21 hours after administration of the myelosuppressive anti-cancer drug; (c) at least 24 hours after administration of the myelosuppressive anti-cancer drug; and (d) at least 27 hours after administration of the myelosuppressive anti-cancer drug.

In another embodiment (E29), the invention provides the method of any one of E 10 to E27, wherein administering recombinant human albumin-human granulocyte colony stimulating factor prior to the myelosuppressive anti-cancer drug induces a rise in WBC.

In another embodiment (E30), the invention provides the method of any one of E 10 to E27, wherein administering recombinant human albumin-human granulocyte colony stimulating factor prior to the myelosuppressive anti-cancer drug induces a rise in ANC.

In another embodiment (E31), the invention provides the method of any one of E22 to E30, wherein the non-myeloid malignancy is a solid tumor.

In another embodiment (E32), the invention provides the method of any one of E10 to E31, wherein the myelosuppressive anti-cancer drug comprises Vincristine, Doxorubicin, Cyclophosphamide, Ifosfamide and Etoposide (VDC/IE) or Vincristine, Ifosfamide, Doxorubicin, and Etoposide (VIDE) chemotherapy regime.

In another embodiment (E33), the invention provides the method of E32, wherein the VIDE chemotherapy regime comprises about 1.5 mg/m² Vincristine, about 3.0 g/m² Ifosfamide, about 20 mg/m² Doxorubicin, and about 150 mg/m² Etoposide for at least one treatment cycle.

In another embodiment (E34), the invention provides the method of E32, wherein the VDC/IE chemotherapy regime comprises about 2.0 mg/m² Vincristine, about 75 mg/m² Doxorubicin, about 1200 mg/m² Cyclophosphamide, about 1800 mg/m²/day Ifosfamide, and about 100 mg/m²/day Etoposide for at least one treatment cycle.

In another embodiment (E35), the invention provides the method of E33, wherein about 1.5 mg/m² Vincristine is given on Day 1, about 3.0 g/m² Ifosfamide is given on Day 1, Day 2, and Day 3, about 20 mg/m² Doxorubicin is given on Day 1, Day 2, and Day 3, and about 150 mg/m² Etoposide is given on Day 1, Day 2, and Day 3 of the treatment cycle for at least one treatment cycle.

In another embodiment (E36), the invention provides the method of E34, wherein about 2.0 mg/m² Vincristine is given on Day 1, Day 8, and Day 15, about 75 m g/m² Doxorubicin is given on Day 1, about 1200 mg/m² Cyclophosphamide is given on Day 1 and Day 2 of a treatment cycle, and about 1800 mg/m²/day Ifosfamide and about 100 mg/m²/day Etoposide are given for 5 days of a treatment cycle for at least one treatment cycle.

In another embodiment (E37), the invention provides the method of E34, wherein about 2.0 mg/m² Vincristine is given on Day 1, Day 8, and Day 15, about 75 mg/m² Doxorubicin is given on Day 1, about 1200 mg/m² Cyclophosphamide is given on Day 1 and Day 2 on chemotherapy treatment cycles 1 and 3, and about 1800 mg/m²/day Ifosfamide and about 100 mg/m²/day Etoposide are given for 5 days on treatment cycles 2 and 4.

In another embodiment (E38), the invention provides the method of E33 or E35, wherein recombinant human albumin-human granulocyte colony stimulating factor is administered at a time selected from the group consisting of: (a) at least 18 hours after administration of Ifosfamide, Doxorubicin or Etoposide; (b) at least 21 hours after administration of Ifosfamide, Doxorubicin or Etoposide; (c) at least 24 hours after administration of Ifosfamide, Doxorubicin or Etoposide; and (d) at least 27 hours after administration of Ifosfamide, Doxorubicin or Etoposide.

In another embodiment (E39), the invention provides the method of any one of E34, E36, or E37, wherein recombinant human albumin-human granulocyte colony stimulating factor is administered at a time selected from the group consisting of: (a) at least 18 hours after administration of Cyclophosphamide; (b) at least 21 hours after administration of Cyclophosphamide; (c) at least 24 hours after administration of Cyclophosphamide; and (d) at least 27 hours after administration of Cyclophosphamide.

In another embodiment (E40), the invention provides the method of any one of E34, E36, E37, or E39 wherein recombinant human albumin-human granulocyte colony stimulating factor is administered at a time selected from the group consisting of: (a) at least 18 hours after administration of Ifosfamide and Etoposide; (b) at least 21 hours after administration of Ifosfamide and Etoposide; (c) at least 24 hours after administration of Ifosfamide and Etoposide; and (d) at least 27 hours after administration of Ifosfamide and Etoposide.

In another embodiment (E41), the invention provides the method of E32, wherein the myelosuppressive anti-cancer drug comprises a VDC/IE chemotherapy regime. In another embodiment (E42), the invention provides the method of E32, wherein the myelosuppressive anti-cancer drug comprises a VIDE chemotherapy regime.

In another embodiment (E43), the invention provides the method of any one of E10 to E31, wherein the myelosuppresive anti-cancer drug comprises an Ifosfamide, Vincristine and Actinomycin D (IVA) chemotherapy regime. In another embodiment (E44), the invention provides the method of E43, wherein the IVA chemotherapy regime comprises about 3.0 g/m² Ifosfamide, about 1.5 mg/m² Vincristine, and about 1.5 mg/m² Actinomycin D for at least one treatment cycle. In another embodiment (E45), the invention provides the method of E44, wherein about 3.0 g/m² Ifosfamide is given on Day 1 and Day 2, about 1.5 mg/m² Vincristine is given on Day 1, Day 8, and Day 15, and 1.5 mg/m² Actinomycin D is given on Day 1 of at least one treatment cycle. In another embodiment (E46), the invention provides the method of E45, wherein about 3.0 g/m² Ifosfamide is given on Day 1 and Day 2, about 1.5 mg/m² Vincristine is given on Day 1, Day 8, and Day 15, and about 1.5 mg/m² Actinomycin D is given on Day 1 of at least treatment Cycles 1, 2, and 4, and about 3.0 g/m² Ifosfamide is given on Day 1 and Day 2, about 1.5 mg/m² Vincristine is given on Day 1, and about 1.5 mg/m² Actinomycin D is given on Day 1 of at least treatment Cycle 3.

In another embodiment (E47), the invention provides the method of any one of E43 to E46, wherein recombinant human albumin-human granulocyte colony stimulating factor is administered at a time selected from the group consisting of: (a) at least 18 hours after administration of Ifosfamide; (b) at least 21 hours after administration of Ifosfamide; (c) at least 24 hours after administration of Ifosfamide; and (d) at least 27 hours after administration of Ifosfamide.

In another embodiment (E48), the invention provides the method of any one of E10 to E31, wherein the myelosuppresive anti-cancer drug comprises an Ifosfamide, Vincristine, Actinomycin D, and Doxorubicin (IVAd) chemotherapy regime. In another embodiment (E49), the invention provides the method of E48, wherein the IVAd chemotherapy regime comprises about 3.0 g/m² Ifosfamide, about 1.5 mg/m² Vincristine, about 1.5 mg/m² Actinomycin D, and about 20 mg/m² Doxorubicin. In another embodiment (E50), the invention provides the method of E49, wherein about 3.0 g/m² Ifosfamide is given on Day 1 and Day 2, about 1.5 mg/m² Vincristine is given on Day 1, Day 8, and Day 15, and about 20 mg/m² Doxorubicin is given twice daily on Day 1 and Day 2 of at least one treatment cycle; and about 3.0 g/m² Ifosfamide is given on Day 1 and Day 2, about 1.5 mg/m² Vincristine is given on Day 1, Day 8, and Day 15, and about 1.5 mg/m² Actinomycin D is given on Day 1 of at least one treatment cycle. In another embodiment (E51), the invention provides the method of E50, wherein about 3.0 g/m² Ifosfamide is given on Day 1 and Day 2, about 1.5 mg/m² Vincristine is given on Day 1, Day 8, and Day 15, and about 20 mg/m² Doxorubicin is given twice daily on Day 1 and Day 2 of at lease treatment Cycles 1 and 4; about 3.0 g/m² Ifosfamide is given on Day 1 and Day 2, about 1.5 mg/m² Vincristine is given on Day 1, Day 8, and Day 15, and about 1.5 mg/m² Actinomycin D is given on Day 1 of at least treatment Cycle 2; and about 3.0 g/m² Ifosfamide is given on Day 1 and Day 2, about 1.5 mg/m² Vincristine is given on Day 1 and about 20 mg/m² Doxorubicin is given twice daily on Day 1 and Day 2 of at least treatment Cycle 3.

In another embodiment (E52), the invention provides the method of any one of E48 to E51, wherein recombinant human albumin-human granulocyte colony stimulating factor is administered at a time selected from the group consisting of: (a) at least 18 hours after administration of Ifosfamide or Doxorubicin; (b) at least 21 hours after administration of Ifosfamide or Doxorubicin; (c) at least 24 hours after administration of Ifosfamide or Doxorubicin; and (d) at least 27 hours after administration of Ifosfamide or Doxorubicin.

In another embodiment (E53), the invention provides the method of any one of E10 to E52, wherein ANC and WBC return to normal at a time period selected from the group consisting of: (a) by day 4 after administration of the anti-cancer drug; (b) by day 5 after administration of the anti-cancer drug; (c) by day 6 after administration of the anti-cancer drug; (d) by day 7 after administration of the anti-cancer drug; (e) by day 8 after administration of the anti-cancer drug; (f) by day 9 after administration of the anti-cancer drug; (g) by day 10 after administration of the anti-cancer drug; (h) by day 11 after administration of the anti-cancer drug; (i) by day 12 after administration of the anti-cancer drug; (j) by day 13 after administration of the anti-cancer drug; (k) by day 14 after administration of the anti-cancer drug; and (1) by day 15 after administration of the anti-cancer drug.

In another embodiment (E54), the invention provides the method of any one of E1 to E53, wherein administering recombinant human albumin-human granulocyte colony stimulating factor induces a rise in lymphocytes, monocytes, eosinophils, basophils, or any combination thereof. In another embodiment (E55), the invention provides the method of any one of E1 to E54, wherein a decrease in the number of lymphocytes, monocytes, eosinophils, or basophils is inhibited in the subject. In another embodiment (E56), the invention provides the method of any one of E1 to E55, wherein said recombinant human albumin-human granulocyte colony stimulating factor is administered in a composition comprising sodium phosphate, mannitol, trehalose dihydrate, polysorbate 80 or a combination thereof.

In another embodiment (E57), the invention provides the method of E56, wherein said recombinant human albumin-human granulocyte colony stimulating factor is administered in a composition comprising 20 mM sodium phosphate, 180 mM mannitol, 60 mM trehalose dihydrate, 0.06% (w/v) polysorbate 80 or a combination thereof. In another embodiment (E58), the invention provides the method of E56 or E57, wherein the composition is at a pH of 5.8-6.2. In another embodiment (E59), the invention provides the method of E56 or E57, wherein the composition is at a pH of about 6.0.

In another embodiment (E60), the invention provides the method of any one of E1 to E60, wherein the recombinant human albumin-human granulocyte stimulating factor is administered parenterally. In another embodiment (E61), the invention provides the method of any one of E1 to E60, wherein the recombinant human albumin-human granulocyte stimulating factor is administered by subcutaneous injection. In another embodiment (E62), the invention provides the method of any one of E1 to E61, wherein the composition is in the form of a lyophilized cake prior to administration.

In another embodiment (E63), the invention provides the method of any one of E10 to E62, wherein the recombinant human albumin-human granulocyte colony stimulating factor is administered once per anti-cancer drug cycle.

In another embodiment (E64), the invention provides the method of any one of E1 to E63, wherein survival rates are improved. In another embodiment (E65), the invention provides the method of any one of E1 to E64, wherein tumor progression is decreased.

In another embodiment (E66), the invention provides the method of any one of E1 to E65, wherein the human subject is receiving a chemotherapeutic agent, and the recombinant human albumin-human granulocyte stimulating factor is administered no more than once per chemotherapeutic cycle.

In another embodiment (E67), the invention provides the method of any one of E1 to E66, wherein the administration of recombinant human albumin-human granulocyte stimulating factor produces an AUC of about 1,000 to about 400,000 ng/mL*hr. In another embodiment (E68), the invention provides the method of any one of E1 to E67, wherein the administration of recombinant human albumin-human granulocyte stimulating factor produces a Cmax of about 25 to about 2000 ng/mL. In another embodiment (E69), the invention provides the method of any one of E1 to E68, wherein the elimination half-life of recombinant human albumin-human granulocyte stimulating factor is about 10 to about 75 hours.

### BRIEF DESCRIPTION OF THE DRAWINGS

FIG. 1A-1C: FIG. 1A shows the nucleic acid sequence (SEQ ID NO:1) and the amino acid sequence (SEQ ID NO:2) of the recombinant human albumin-granulocyte colony stimulating factor (“rHA-G-CSF”) fusion protein termed “Neugranin™” (“NEUG”); FIG. 1B shows the amino acid sequence of human G-CSF (SEQ ID NO:3); FIG. 1C shows the amino acid sequence of human serum albumin (SEQ ID NO:4).

FIG. 2 shows the overall study scheme described in Example 1.

### DETAILED DESCRIPTION

The present invention discloses methods and compositions useful for the treatment, amelioration and prevention of conditions characterized by a lower than normal white blood cell count. Such conditions include but are not limited to leukopenia and neutropenia.

In a first embodiment, described is a method of treating or preventing neutropenia in a human subject comprising administering to a human subject exhibiting neutropenia or at risk of developing neutropenia, recombinant human albumin-human granulocyte colony stimulating factor in an amount effective to treat the subject wherein the subject is less than 18 years old. In an exemplary embodiment, the human subject can be suffering from a non-myeloid malignancy and receiving at least one myelosuppressive anti-cancer drug associated with a clinically significant incidence of febrile neutropenia.

According to one aspect, the present invention relates to a method of decreasing the incidence of infection, as manifested by febrile neutropenia, in a human subject with non-myeloid malignancies and receiving at least one myelosuppressive anti-cancer drug associated with a clinically significant incidence of febrile neutropenia, comprising administering to the human subject recombinant human albumin-human granulocyte colony stimulating factor in an amount effective to treat the subject wherein the subject is less than 18 years old.

In one embodiment, the human subject receiving the recombinant human albumin-human granulocyte colony stimulating factor is less than two years old. In another embodiment the subject receiving the recombinant human albumin-human granulocyte colony stimulating factor is one month to 18 years old. In yet another embodiment the subject receiving the recombinant human albumin-human granulocyte colony stimulating factor is 2-11 years old. In yet another embodiment the subject receiving the recombinant human albumin-human granulocyte colony stimulating factor is 12-17 years old. In yet another embodiment the subject receiving the recombinant human albumin-human granulocyte colony stimulating factor is 2-17 years old. According to one embodiment the subject is a male. According to another embodiment, the subject is a female. According to one embodiment the subject is a male or a female who is less than 18 years old.

According to another aspect of the invention, the human subject weighs less than 60 Kg, less than 50 Kg less than 40 Kg, less than 30 Kg, less than 20 Kg. According to yet another aspect of the invention the human subject weighs less than 29.8 Kg. In another aspect of the invention, the human subject weighs about 5 to about 60 Kg, about 5 to about 50 Kg, about 5 to about 40 Kg, about 5 to about 30 Kg, about 5 to about 20 Kg or about 5 to about 10 Kg. In another aspect of the invention, the human subject weighs about 10 to about 60 Kg, about 10 to about 50 Kg, about 10 to about 40 Kg, about 10 to about 30 Kg, or about 10 to 20 Kg. In another aspect of the invention, the human subject weighs about 15 to about 60 Kg, about 15 to about 50 Kg, about 15 to about 40 Kg, or about 15 to about 30 Kg. In another aspect of the invention, the human subject weighs about 20 to about 60 Kg, about 20 to about 50 Kg, about 20 to about 40 Kg, or about 20 to about 30 Kg.

In some embodiments, the compositions and methods include a fusion polypeptide formed from human serum albumin protein (“HSA”) and human granulocyte-colony stimulating factor (“G-CSF”). The fusion polypeptide can be 759 amino acids in length, wherein amino acids 1-585 of the fusion correspond to amino acids from the mature form of HSA, and amino acids 586-759 of the fusion correspond to amino acids of the mature form of human G-CSF. The amino acid sequences of such fusion proteins are presented in FIG. 1. The fusion polypeptide, termed Neugranin™ (“NEUG”) is administered to patients that are 2-17 years old exhibiting or at risk of exhibiting leukopenia or neutropenia. For example, in some embodiments, methods include treating leukopenia or neutropenia in a human subject that is less than 18 years old by administering recombinant human albumin-human granulocyte colony stimulating factor in an amount effective to treat the subject.

In other embodiments, methods include decreasing the incidence of infection, as manifested by febrile neutropenia, in a human subject that is less than 18 years old. In some embodiments, the human subject is suffering from a non-myeloid malignancy and is receiving at least one myelosuppressive anti-cancer drug associated with a clinically significant incidence of febrile neutropenia. In some embodiments, recombinant human albumin-human granulocyte colony stimulating factor is administered to the subject in an amount effective to treat the neutropenia in the subject that is less than 18 years old. In yet another embodiment of the invention, the non-myeloid malignancy is a solid tumor, lymphoma, neuroblastoma, Wilm's tumor, sarcoma, medullobastoma, Ewing's sarcoma, Rhabdomyosarcoma, other soft tissue sarcoma. In yet another embodiment of the invention, the non-myeloid malignancy is bladder cancer, breast cancer, eophageal and gastric cancer, non-Hodgkin's lymphoma, melanoma, myelodysplasic syndrome, ovarian cancer, pancreatic cancer, sarcoma, small cell lung cancer, or testicular cancer. In yet another embodiment of the invention, the non-myeloid malignancy is a solid tumor or lymphoma. In yet another embodiment of the invention, the non-myeloid malignancy is a solid tumor.

In some embodiments, the duration or severity of neutropenia is reduced or neutropenia is eliminated in a subject that is less than 18 years old. For example, in some embodiments, grade 4 or grade 3 neutropenia in the subject is eliminated. In other embodiments, the duration of grade 4 or grade 3 neutropenia is reduced. For example, in some embodiments the duration of grade 4 neutropenia in the subject is less than 5 days; in some embodiments, the duration of grade 4 neutropenia in the subject is less than 4 days, less than 3 days or less than 2 days. In other embodiments, the duration of grade 3 neutropenia in the subject is eliminated, and/or the duration of grade 3 neutropenia in the subject is decreased as compared to subjects who do not receive treatment with human albumin-human granulocyte colony stimulating factor. In some embodiments, the duration of severe neutropenia is reduced in the subject. In other embodiments the incidence of severe neutropenia is reduced in the subject. In yet another embodiment, the frequency of febrile neutropenia is reduced in the subject.

Human granulocyte colony stimulating factor induces a rise in white blood cells (“WBC”) or a decrease in loss of WBC in a subject. For example, in some embodiments, the number of neutrophils is increased in the subject; a decrease in the number of neutrophils is inhibited in the subject, the nadir absolute neutrophil count (“ANC”) is increased in the subject, the recovery ANC is increased in the subject, and/or the time to ANC recovery is reduced in the subject.

In some embodiments, the amount of recombinant human albumin-human granulocyte colony stimulating factor administered to the subject is from about 200 μg/kg to about 1000 μg/kg; in other embodiments, the amount of recombinant human albumin-human granulocyte colony stimulating factor administered to the subject is about 200 μg/kg to about 700 μg/kg. In other embodiments, the amount of recombinant human albumin-human granulocyte colony stimulating factor administered to the subject is about 300 μg/kg to about 670 μg/kg. In still other embodiments, the amount of recombinant human albumin-human granulocyte colony stimulating factor administered to the subject is about 200 μg/kg is about 250 μg/kg, about 300 μg/kg, about 500 μg/kg, about 650 μg/kg, about 670 μg/kg or about 700 μg/kg. In yet other embodiments, the amount of recombinant human albumin-human granulocyte colony stimulating factor administered to the subject is from about 0.4 to about 60 mg, from about 0.4 to about 50 mg, from about 0.4 to about 40 mg, from about 0.4 to about 30 mg, or from about 0.4 to about 20 mg. In further embodiments, the amount of recombinant human albumin-human granulocyte colony stimulating factor administered to the subject is from about 3 mg to about 60 mg, from about 3 mg to about 50 mg, from about 3 mg to about 40 mg, from about 3 mg to about 30 mg, or from about 3 mg to about 20 mg. In further embodiments, the amount of recombinant human albumin-human granulocyte colony stimulating factor administered to the subject is from about 10 mg to about 60 mg, from about 10 mg to about 50 mg, from about 10 mg to about 40 mg, from about 10 mg to about 30 mg, or from about 10 mg to about 20 mg. In further embodiments, the amount of recombinant human albumin-human granulocyte colony stimulating factor administered to the subject is from about 20 mg to about 60 mg from about 20 mg to about 50 mg, from about 20 mg to about 40 mg, or from about 20 mg to about 30 mg. In further embodiments, the amount of recombinant human albumin-human granulocyte colony stimulating factor administered to the subject is from about 30 mg to about 60 mg from about 30 mg to about 50 mg, or from about 30 mg to about 40 mg. In further embodiments, the amount of recombinant human albumin-human granulocyte colony stimulating factor administered to the subject is less than 5 mg, less than 10 mg, less than 20 mg, less than 30 mg, less than 40 mg or less than 50 mg. In further embodiments, the amount of recombinant human albumin-human granulocyte colony stimulating factor administered to the subject is about 5 mg, about 10 mg, about 15 mg, about 20 mg, about 25 mg, about 30 mg, about 35 mg, or about 40 mg.

In some embodiments, the neutropenia is primary neutropenia, acute neutropenia, severe chronic neutropenia (SCN), febrile neutropenia, severe congenital neutropenia (Kostmann's syndrome), severe infantile genetic agranulocytosis, benign neutropenia, cyclic neutropenia, chronic idiopathic neutropenia, secondary neutropenia, syndrome associated neutropenia, or immune-mediated neutropenia.

In other embodiments, the neutropenia is caused or associated with radiation, alcoholism, drugs, allergic disorders, aplastic anemia, autoimmune disease, T-γ lymphoproliferative disease (T-γ LPD), myelodysplasia, myelofibrosis, dysgammaglobulinemia, paroxysmal nocturnal hemoglobinuria, cancer, vitamin B₁₂ deficiency, folate deficiency, viral infection, bacterial infection, spleen disorder, hemodialysis, transplantation, leukemia, myeloma, lymphoma, a metastatic solid tumor which infiltrates and replaces the bone marrow, a toxin, bone marrow failure, Schwachman-Diamond syndrome, cartilage-hair hypoplasia, dyskeratosis congenita, glycogen storage disease type IB, splenomegaly of any cause, and an intrinsic defect in myeloid cells or their precursors. In some embodiments, the neutropenia is caused or associated with cytotoxic chemotherapy.

In embodiments, where the human subject is suffering from a non-myeloid malignancy, the non-myeloid malignancy can comprise solid tumors or lymphoma.

In another embodiment, recombinant human albumin-human granulocyte colony stimulating factor can be administered to a patient who has received or will receive a myelosuppressive anti-cancer drug. For example, the recombinant human albumin-human granulocyte colony stimulating factor can be administered at a time selected from the group consisting of: (a) at least 2 hours after administration of the myelosuppressive anti-cancer drug; (b) at least 4 hours after administration of the myelosuppressive anti-cancer drug; (c) at least 6 hours after administration of the myelosuppressive anti-cancer drug; (d) at least 12 hours after administration of the myelosuppressive anti-cancer drug; (e) at least 18 hours after administration of the myelosuppressive anti-cancer drug; (f) at least 21 hours after administration of the myelosuppressive anti-cancer drug; (g) at least 24 hours after administration of the myelosuppressive anti-cancer drug; (g) at least 27 hours after administration of the myelosuppressive anti-cancer drug; (h) at least 48 hours after administration of the myelosuppressive anti-cancer drug; or (i) during, or substantially concurrently with, the administration of the myelosuppressive anti-cancer drug. In some embodiments, the recombinant human albumin-human granulocyte colony stimulating factor is administered within 7, 6, 5, 4, 3, 2, or 1 days after administration of the myelosuppressive anti-cancer drug or on the same day as administration of the myelosuppressive anti-cancer drug. Thus, in some embodiments, the recombinant human albumin-human granulocyte colony stimulating factor is administered at least 2, 4, 6 12, 21, 24, 27, or 48 hours after administration of the myelosuppressive anti-cancer drug and within 7, 6, 5, 4, or 3 days of administration of the myelosuppressive anti-cancer drug. In some embodiments, recombinant human albumin-human granulocyte colony stimulating factor is administered prior to administration of the myelosuppressive anti-cancer drug.

In some embodiments, administering recombinant human albumin-human granulocyte colony stimulating factor during or after chemotherapy treatment induces a rise in WBC and/or induces a rise in ANC. For example, in some embodiments, ANC and/or WBC return to normal by day 4 after chemotherapy. In other embodiments ANC and/or WBC return to normal by day 5 after chemotherapy, by day 6 after chemotherapy, by day 7 after chemotherapy, by day 8 after chemotherapy, by day 9 after chemotherapy, by day 10 after chemotherapy, by day 11 after chemotherapy, by day 12 after chemotherapy, by day 13 after chemotherapy, by day 14 after chemotherapy or by day 15 after chemotherapy. In some embodiments, on day 9, 10, 11, 12, 13, or 14 after chemotherapy administration, the rise in ANC in patients treated with recombinant human albumin-human granulocyte colony stimulating factor is lower than the rise in ANC in patients treated with an equivalent dose of pegfilgrastim.

In some embodiments, on day 9, 10, 11, 12, 13, or 14 after chemotherapy administration, the rise in ANC in patients treated with recombinant human albumin-human granulocyte colony stimulating factor is higher than the rise in ANC in patients treated with an equivalent dose of pegfilgrastim.

In some embodiments, on day 9, 10, 11, 12, 13, or 14 after chemotherapy administration, the rise in ANC in patients treated with recombinant human albumin-human granulocyte colony stimulating factor is similar to the rise in ANC in patients treated with an equivalent dose of pegfilgrastim.

In some embodiments, administration of human albumin-human granulocyte colony stimulating factor to a pediatric patient results in an AUC of about 1,000 to about 400,000 ng/ml*hr, or about 1,000 to about 300,000 ng/ml*hr, about 1,000 to about 200,000 ng/ml*hr, about 1,000 to about 100,000 ng/ml*hr, about 1,000 to about 75,000 ng/ml*hr, or about 1,000 to about 50,000 ng/ml*hr. In some embodiments, administration of human albumin-human granulocyte colony stimulating factor to a pediatric patient results in an AUC of about 2,000 to about 400,000 ng/ml*hr, or about 2,000 to about 300,000 ng/ml*hr, about 2,000 to about 200,000 ng/ml*hr, about 2,000 to about 100,000 ng/ml*hr, about 2,000 to about 75,000 ng/ml*hr, or about 2,000 to about 50,000 ng/ml*hr. In some embodiments, administration of human albumin-human granulocyte colony stimulating factor to a pediatric patient results in an AUC of about 5,000 to about 400,000 ng/ml*hr, or about 5,000 to about 300,000 ng/ml*hr, about 5,000 to about 200,000 ng/ml*hr, about 5,000 to about 100,000 ng/ml*hr, about 5,000 to about 750,000 ng/ml*hr, or about 5,000 to about 500,000 ng/ml*hr.

In some embodiments, administration of 300 μg/kg human albumin-human granulocyte colony stimulating factor to a pediatric patient results in an AUC of about 1,000 to about 100,000 ng/ml*hr, about 1,000 to about 90,000 ng/ml*hr, about 1,000 to about 80,000 ng/ml*hr, about 1,000 to about 75,000 ng/ml*hr, about 1,000 to about 70,000 ng/ml*hr, or about 1,000 to about 60,000 ng/ml*hr, or about 1,000 to about 50,000 ng/ml*hr. In some embodiments, administration of 300 μg/kg human albumin-human granulocyte colony stimulating factor to a pediatric patient results in an AUC of about 2,000 to about 100,000 ng/ml*hr, about 2,000 to about 90,000 ng/ml*hr, about 2,000 to about 80,000 ng/ml*hr, about 2,000 to about 75,000 ng/ml*hr, about 2,000 to about 70,000 ng/ml*hr, or about 2,000 to about 60,000 ng/ml*hr, or about 2,000 to about 50,000 ng/ml*hr.

In some embodiments, administration of 670 μg/kg human albumin-human granulocyte colony stimulating factor to a pediatric patient results in an AUC of about 5,000 to about 400,000 ng/ml*hr, about 5,000 to about 375,000 ng/ml*hr about 5,000 to about 350,000 ng/ml*hr, about 5,000 to about 325,000 ng/ml*hr, about 5,000 to about 300,000 ng/ml*hr, or about 5,000 to about 275,000 ng/ml*hr. In some embodiments, administration of 670 μg/kg human albumin-human granulocyte colony stimulating factor to a pediatric patient results in an AUC of about 7,500 to about 400,000 ng/ml*hr, about 7,500 to about 375,000 ng/ml*hr about 7,500 to about 350,000 ng/ml*hr, about 7,500 to about 325,000 ng/ml*hr, about 7,500 to about 300,000 ng/ml*hr, or about 7,500 to about 275,000 ng/ml*hr.

In some embodiments, administration of human albumin-human granulocyte colony stimulating factor to a pediatric patient results in a Cmax of about 25 to about 2000 ng/mL or about 25 to about 1500 ng/mL. In some embodiments, administration of 300 μg/kg human albumin-human granulocyte colony stimulating factor to a pediatric patient results in a Cmax of about 25 to about 1400 ng/mL, about 25 to about 1300 ng/mL, about 25 to about 1200 ng/mL, about 25 to about 1100 ng/mL, about 25 to about 1000 ng/mL, about 25 to about 900 ng/mL, about 25 to about 800 ng/mL, or about 25 to about 750 ng/mL. In some embodiments, administration of 300 μg/kg human albumin-human granulocyte colony stimulating factor to a pediatric patient results in a Cmax of about 30 to about 1400 ng/mL, about 30 to about 1300 ng/mL, about 30 to about 1200 ng/mL, about 30 to about 1100 ng/mL, about 30 to about 1000 ng/mL, about 30 to about 900 ng/mL, about 30 to about 800 ng/mL, or about 30 to about 750 ng/mL. In some embodiments, administration of 670 μg/kg human albumin-human granulocyte colony stimulating factor to a pediatric patient results in a Cmax of about 40 to about 2000 ng/mL, about 40 to about 1900 ng/mL, about 40 to about 1800 ng/mL, about 40 to about 1700 ng/mL, about 40 to about 1600 ng/mL, or about 40 to about 1500 ng/mL.

In some embodiments, the elimination half-life of human albumin-human granulocyte colony stimulating factor in a pediatric patient is about 10 to about 75 hours, about 10 to about 50 hours, about 10 to about 30 hours, about 30 to about 50 hours, or about 30 to about 75 hours. In some embodiments, the elimination of a dose of 300 μg/kg human albumin-human granulocyte colony stimulating factor is about 10 to about 75 hours. In some embodiments, the elimination of a dose of 300 μg/kg human albumin-human granulocyte colony stimulating factor is about 10 to about 75 hours or about 10 to about 70 hours.

In embodiments of the invention the human subject is suffering from a non-myeloid malignancy. The non-myeloid malignancy can comprise solid tumors or lymphoma.

In some embodiments, the human subject that is less than 18 years old is suffering from a solid tumor, lymphoma, neuroblastoma, Wilm's tumor, sarcoma, medullobastoma, Ewing's sarcoma, Rhabdomyosarcoma, or other soft tissue sarcoma, and is receiving cytotoxic chemotherapy. In some embodiments, the human subject that is less than 18 years old is suffering from bladder cancer, breast cancer, eophageal and gastric cancer, non-Hodgkin's lymphoma, melanoma, myelodysplasic syndrome, ovarian cancer, pancreatic cancer, sarcoma, small cell lung cancer, or testicular cancer. In some embodiments, the human subject that is less than 18 years old is suffering from a solid tumor, lymphoma, neuroblastoma, Wilm's tumor, sarcoma, medullobastoma, Ewing's sarcoma, Rhabdomyosarcoma, or other soft tissue sarcoma, and is receiving chemotherapy. In some embodiments, the human subject that is less than 18 years old is suffering from a bladder cancer, breast cancer, eophageal and gastric cancer, non-Hodgkin's lymphoma, melanoma, myelodysplasic syndrome, ovarian cancer, pancreatic cancer, sarcoma, small cell lung cancer, or testicular cancer, and is receiving chemotherapy. In some embodiments, the human subject that is less than 18 years old is suffering from a non-myeloid malignancy, for example, a solid tumor or lymphoma, and is receiving cytotoxic chemotherapy. For example, in some embodiments the patient is receiving at least one myelosuppressive anti-cancer drug associated with a clinically significant incidence of febrile neutropenia.

In some embodiments, the myelosuppressive anticancer drug comprises Vincristine, Ifosfamie, Doxorubicin, Etoposide, Cyclophosphamide, Actinomycin D, or a combination thereof. In some embodiments, the myelosuppressive anticancer drug comprises Vincristine, Ifosfamie, Doxorubicin, and Etoposide. In some embodiments, the myelosuppressive anticancer drug comprises Vincristine, Ifosfamie, Doxorubicin, Etoposide, and Cyclophosphamide. In some embodiments, the myelosuppressive anticancer drug comprises Vincristine, Ifosfamie, and Actinomycin D. In some embodiments, the myelosuppressive anticancer drug comprises Vincristine, Ifosfamie, Actinomycin D, and Doxorubicin.

In some embodiments, the myelosuppressive anticancer drugs comprise a an Ifosfamide, Vincristine and Actinomycin D (IVA) chemotherapy regime or an Ifosfamide, Vincristine, Actinomycin D, and Doxorubicin chemotherapy regime (IVAd).

In some embodiments, the myelosuppressive anticancer drugs comprise a Vincristine, Doxorubicin, Cyclophosamide, Ifosfamide, and Etoposide (VDC/IE) chemotherapy regime or a Vincristine, Ifosfamide, Doxorubicin, and Etoposide (VIDE) chemotherapy regime. In further embodiments, the VIDE chemotherapy regime comprises about 1.5 mg/m² Vincristine, about 3.0 g/m² Ifosfamide, about 20 mg/m² Doxorubicin and about 150 mg/m² Etoposide for at least one treatment cycle. In further embodiments the VIDE chemotherapy regime comprises about 1.5 mg/m² Vincristine given on Day 1, about 3.0 g/m² Ifosfamide given on Day 1, Day 2, and Day 3, about 20 mg/m² Doxorubicin given on Day 1, Day 2, and Day 3 and about 150 mg/m² Etoposide given on Day 1, Day 2, and Day 3 of the treatment cycle for at least one treatment cycle. In some embodiments, the VIDE regimen is administered by intravenous infusion. In some embodiments, the VIDE regimen is administered orally, e.g., in a tablet. In yet another embodiment the VDC/IE regime comprises about 2.0 mg/m² Vincristine, about 75 mg/m² Doxorubicin, about 1200 mg/m² Cyclophosphamide, about 1800 mg/m²/day Ifosfamide and about 100 mg/m²/day Etoposide for at least one treatment cycle. In yet another embodiment, the VDC/IE regime comprises about 2.0 mg/m² Vincristine given on Day 1, Day 8, and Day 15, about 75 mg/m² Doxorubicin given on Day 1, about 1200 mg/m² Cyclophosphamide given on Day 1 and Day 2, about 1800 mg/m²/day Ifosfamide and about 100 mg/m²/day Etoposide given for 5 days for at least one treatment cycle. In some embodiments, the VDC/IE regimen is administered by intravenous infusion. In some embodiments, the VDC/IE regimen is administered orally, e.g., in a tablet.

In yet another embodiment of the invention the VDC/IE regime comprises about 2.0 mg/m² Vincristine given on Day 1, Day 8, and Day 15, about 75 mg/m² Doxorubicin given on Day 1, and about 1200 mg/m² Cyclophosphamide given on Day land Day 2 on chemotherapy treatment cycles 1 and 3, and about 1800 mg/m²/day Ifosfamide and about 100 mg/m²/day Etoposide given for 5 days on treatment cycles 2 and 4. In some embodiments, the VDC/IE regimen is administered by intravenous infusion. In some embodiments, the VDC/IE regimen is administered orally, e.g., in a tablet.

In yet another embodiment, the recombinant human albumin-human granulocyte colony stimulating factor is administered in the VIDE chemotherapy regime at a time selected from the group consisting of: (a) at least 18 hours after administration of Ifosfamide, Doxorubicin or Etoposide; (b) at least 21 hours after administration of Ifosfamide, Doxorubicin or Etoposide; (c) at least 24 hours after administration of Ifosfamide, Doxorubicin or Etoposide; or (d) at least 27 hours after administration of Ifosfamide, Doxorubicin or Etoposide. In one exemplary embodiment, the recombinant human albumin-human granulocyte colony stimulating factor is administered in the VIDE chemotherapy regime at a time selected from the group consisting of: (a) at least 18 hours after administration of Ifosfamide, Doxorubicin or Etoposide at the end of the last chemotherapy administration in Week 1 of treatment cycle; (b) at least 21 hours after administration of Ifosfamide, Doxorubicin or Etoposide at the end of the last chemotherapy administration in Week 1 of treatment cycle; (c) at least 24 hours after administration of Ifosfamide, Doxorubicin or Etoposide at the end of the last chemotherapy administration in Week 1 of treatment cycle or (d) at least 27 hours after administration of Ifosfamide, Doxorubicin or Etoposide at the end of the last chemotherapy administration in Week 1 of treatment cycle.

In an embodiment of the invention, the VDC/IE chemotherapy regime comprises about 2.0 mg/m² Vincristine, about 75 mg/m² Doxorubicin, about 1200 mg/m² Cyclophosphamide, about 1800 mg/m²/day Ifosfamide and about 100 mg/m²/day Etoposide for at least one treatment cycle. In another embodiment, the VDC/IE chemotherapy regime comprises about 2.0 mg/m² Vincristine given on Day 1, Day 8, and Day 15, about 75 mg/m² Doxorubicin given on Day 1, about 1200 mg/m² Cyclophosphamide given on Day 1 and Day 2 of treatment cycle, and about 1800 mg/m²/day Ifosfamide and about 100 mg/m²/day Etoposide given for 5 days of treatment cycle for at least one treatment cycle. In yet another embodiment, the VDC/IE chemotherapy regime comprises about 2.0 mg/m² Vincristine given on Day 1, Day 8, and Day 15, about 75 mg/m² Doxorubicin given on Day 1, about 1200 mg/m² Cyclophosphamide given on Day 1 and Day 2 on chemotherapy treatment cycles 1 and 3, and about 1800 mg/m²/day Ifosfamide and about 100 mg/m²/day Etoposide given for 5 days by intravenous infusion on treatment cycles 2 and 4.

According to an embodiment, the recombinant human albumin-human granulocyte colony stimulating factor is administered in the VDC/IE chemotherapy regime at a time selected from the group consisting of: (a) at least 18 hours after administration of Cyclophosphamide; (b) at least 21 hours after administration of Cyclophosphamide; (c) at least 24 hours after administration of Cyclophosphamide; and (d) at least 27 hours after administration of Cyclophosphamide. In one exemplary embodiment, the recombinant human albumin-human granulocyte colony stimulating factor is administered in the VDC/IE chemotherapy regime at a time selected from the group consisting of: (a) at least 18 hours after administration of Cyclophosphamide at the end of the last chemotherapy administration in Week 1 of treatment cycle; (b) at least 21 hours after administration of Cyclophosphamide at the end of the last chemotherapy administration in Week 1 of treatment cycle; (c) at least 24 hours after administration of Cyclophosphamide at the end of the last chemotherapy administration in Week 1 of treatment cycle; and (d) at least 27 hours after administration of Cyclophosphamide at the end of the last chemotherapy administration in Week 1 of treatment cycle.

VDC/IE and VIDE chemotherapy regimes can be administered to adult or pediatric human subjects. Thus, in some embodiments, methods of treating or preventing neutropenia are provided, wherein the treatment comprises administering to a human subject receiving Vincristine, Ifosfamide, Doxorubicin, Etoposide, Cyclophosphamide, and/or Actinomycin D (e.g., VDC/IE, VIDE, IVA, or IVAd) recombinant human albumin-human granulocyte stimulating factor in an amount effective to treat the subject. In some embodiments, the human albumin-human granulocyte stimulating factor is administered about 21, about 24, or about 27 hours after the Vincristine, Ifosfamide, Doxorubicin, Etoposide, Cyclophosphamide or Actinomycin D. In some embodiments, the human albumin-human granulocyte stimulating factor is administered at least 21, at least 24, or at least 27 hours after the Vincristine, Ifosfamide, Doxorubicin, Etoposide, Cyclophosphamide or Actinomycin D. In some embodiments, the human albumin-human granulocyte stimulating factor is administered prior to administration of the Vincristine, Ifosfamide, Doxorubicin, Etoposide, Cyclophosphamide or Actinomycin D.

In addition embodiments, methods of decreasing the incidence of infection as manifested by febrile neutropenia in a human subject receiving at least one of Vincristine, Ifosfamide, Doxorubicin, Etoposide, Cyclophosphamide, and Actinomycin D (e.g., VDC/IE, VIDE, IVA, or IVAd) are provided, wherein the methods comprise administering recombinant human albumin-human granulocyte stimulating factor to the subject in an amount effect to treat the subject. In some embodiments, the human albumin-human granulocyte stimulating factor is administered about 21, about 24, or about 27 hours after the Vincristine, Ifosfamide, Doxorubicin, Etoposide, Cyclophosphamide or Actinomycin D. In some embodiments, the human albumin-human granulocyte stimulating factor is administered at least 21, at least 24, or at least 27 hours after the Vincristine, Ifosfamide, Doxorubicin, Etoposide, Cyclophosphamide or Actinomycin D. In some embodiments, the human albumin-human granulocyte stimulating factor is administered prior to administration of the Vincristine, Ifosfamide, Doxorubicin, Etoposide, Cyclophosphamide or Actinomycin D.

In some embodiments, administering recombinant human albumin-human granulocyte colony stimulating factor induces a rise in lymphocytes, monocytes, eosinophils, basophils, or any combination thereof. In other embodiments, the number of lymphocytes, monocytes, eosinophils, basophils or any combination thereof is increased in the subject by, for example, 5%, 10%, 15%, 20%, 25%, 30%, 35%, 40%, 45%, 50% or more. In yet further embodiments, a decrease in the number of lymphocytes, monocytes, eosinophils, or basophils is inhibited in the subject.

According to one aspect, a method comprising administering an effective amount of a composition comprising recombinant human albumin-human granulocyte colony stimulating factor and an acceptable pharmaceutical carrier to a human subject less than 18 years old is described. According to another aspect, the composition comprises the recombinant human albumin-human granulocyte colony stimulating factor and an acceptable pharmaceutical carrier selected from sodium phosphate, mannitol, trehalose dihydrate, polysorbate 80 and a combination thereof. According to yet another aspect, the composition comprises the recombinant human albumin-human granulocyte colony stimulating factor and an acceptable pharmaceutical carrier selected from 20 mM sodium phosphate, 180 mM mannitol, 60 mM trehalose dihydrate, 0.06% (w/v) polysorbate 80 and a combination thereof. According to one embodiment, the composition comprising the recombinant human albumin-human granulocyte colony stimulating factor and an acceptable pharmaceutical carrier is at a pH of about 5.8 to about 6.2. According to another embodiment, the composition comprising the recombinant human albumin-human granulocyte colony stimulating factor and an acceptable pharmaceutical carrier is at a pH of about 6.0.

In some embodiments, the composition comprises a concentration of recombinant human albumin-human granulocyte colony stimulating factor of about 30 to about 120 mg/ml, about 30 to about 100 mg/ml, about 30 to about 90 mg/ml, about 30 to about 80 mg/ml or about 30 to about 75 mg/ml. In some embodiments, the composition comprises a concentration of recombinant human albumin-human granulocyte colony stimulating factor of about 40 to about 120 mg/ml, about 40 to about 100 mg/ml, about 40 to about 90 mg/ml, about 40 to about 80 mg/ml, or about 40 to about 75 mg/ml. In some embodiments, the composition comprises a concentration of recombinant human albumin-human granulocyte colony stimulating factor of about 40 to about 60 mg/ml. In some embodiments, the composition comprises a concentration of recombinant human albumin-human granulocyte colony stimulating factor of about 50 mg/ml.

In an embodiment, the composition is formulated for parenteral administration.

In an embodiment, the composition is administered by subcutaneous injection.

In an embodiment, the composition is in the form of a lyophilized cake prior to administration.

### I. DEFINITIONS

As used herein, “polynucleotide” refers to a nucleic acid molecule and includes, for example, a nucleotide sequence encoding a fusion protein comprising, consisting essentially of, or consisting of, at least one molecule of albumin (or a fragment or variant thereof) joined in frame to at least one molecule of Granulocyte-colony stimulating factor (G-CSF) (or fragment or variant thereof). As used herein, “albumin fusion construct” refers to a nucleic acid molecule comprising, or alternatively consisting of, a polynucleotide encoding at least one molecule of albumin (or a fragment or variant thereof) joined in frame to at least one polynucleotide encoding another polypeptide, for example, G-CSF (or fragment or variant thereof). The albumin fusion construct can further comprise, for example, one or more of the following elements: (1) a functional self-replicating vector (including but not limited to, a shuttle vector, an expression vector, an integration vector, and/or a replication system), (2) a region for initiation of transcription (e.g., a promoter region, such as for example, a regulatable or inducible promoter, a constitutive promoter), (3) a region for termination of transcription, (4) a leader sequence, and (5) a selectable marker. The polynucleotides encoding the G-CSF and albumin proteins, once part of the albumin fusion construct, can each be referred to as a “portion,” “region” or “moiety” of the albumin fusion construct.

By a G-CSF polypeptide displaying a “therapeutic activity” or a G-CSF protein that is “therapeutically active” is meant a G-CSF polypeptide that possesses one or more known biological and/or therapeutic activities associated with G-CSF protein. As a non-limiting example, a “G-CSF therapeutic protein” is a G-CSF protein that is useful to treat, prevent or ameliorate a disease, condition or disorder. As a non-limiting example, a “G-CSF therapeutic protein” can be one that binds specifically to a particular cell type (normal (e.g., lymphocytes) or abnormal e.g., (cancer cells)) and therefore can be used to target a compound (drug, or cytotoxic agent) to that cell type specifically.

### II. GRANULOCYTE-COLONY STIMULATING FACTOR

Granulocyte-colony stimulating factor (G-CSF) is a hematopoietic growth factor that stimulates the production of neutrophils. Administration of G-CSF results in rapid induction of a neutrophilic leukocytosis when there are viable precursor cells to stimulate. Another important in vivo activity of G-CSF is mobilization of hematopoietic progenitor cells into the peripheral blood (Dürhsen U. et al. Blood; 72:2074-2080 (1988)). This effect includes not only the neutrophil lineage but extends to other single lineage and multi-lineage progenitors and pluripotent hematopoietic stem cells. G-CSF also increases the cellular events that are part of the defense mechanism against infections by priming neutrophils, thereby increasing both their phagocytic and anti-bacterial activities against opsonized Staphylococcus aureus. G-CSF also induces chemotaxis of neutrophils and monocytes and adhesion of neutrophils.

Primary prophylaxis with G-CSF can be used for the prevention of febrile neutropenia in patients who are at high risk based on age, medical history, disease characteristics, and myelotoxicity of the chemotherapy regimen. The American Society of Clinical Oncology and the European Organization for Research and Treatment of Cancer recommend the use of G-CSF when the risk of febrile neutropenia is approximately 20%. The U.S. National Comprehensive Cancer Center Network recommends an optional indication of G-CSF prophylaxis when the risk of febrile neutropenia is 10% to 20% and a definite indication of G-CSF prophylaxis when the risk of febrile neutropenia is at least 20%.

Prophylaxis with colony-stimulating factors can be used to alleviate the toxicity of certain chemotherapy regimens. The G-CSF protein can comprise the sequence of a native human G-CSF protein. The native human G-CSF protein can be encoded by a wild type polynucleotide sequence (e.g., either full length or mature), or in some instances the sequence can be encoded by a variant of the wild type polynucleotide sequence (e.g., a polynucleotide which encodes the wild type G-CSF protein, wherein the DNA sequence of the polynucleotide has been optimized, for example, for expression in a particular species such as yeast). In another embodiment, the G-CSF protein is encoded by or a polynucleotide encoding a variant of the wild type G-CSF protein (i.e., a site directed mutant). In some embodiments, the G-CSF protein is encoded by an allelic variant found in humans.

### III. HUMAN SERUM ALBUMIN

Human serum albumin (HSA or HA) is the most prevalent naturally occurring blood protein in the human circulatory system, measured at approximately 40 grams of albumin/liter and persisting in the circulation for over 20 days. Albumin is a carrier protein with minimal activity at physiological concentrations. Even though HSA lacks enzymatic or immunological function, it is widely distributed in vivo, and is know to be a carrier for various substances in the blood (e.g., hormones, fatty acids, unconjugated bilirubin, etc. Both HSA and recombinant HA (rHA) have the same long circulating half-life in humans.

Research has shown that therapeutic proteins genetically fused to human albumin are able to take on the circulating half-life characteristics of albumin. For example, in rabbits, the half-life of CD4 fused to albumin is 140 fold greater than non-fused CD4.

Human serum albumin, a protein of 585 amino acids in its mature form (as shown in FIG. 1), is responsible for a significant proportion of the osmotic pressure of serum and also functions as a carrier of endogenous and exogenous ligands. HA for clinical can be produced by extraction from human blood. HA can also be produced recombinantly, and the production of recombinant HA (rHA) in microorganisms has been disclosed, for example, in EP 330 451 and EP 361 991.

### IV. POLYPEPTIDE AND POLYNUCLEOTIDE FRAGMENTS AND VARIANTS

Fragments of G-CSF protein, albumin proteins, and/or albumin fusion proteins can also be used according to the methods provided herein. Polynucleotides encoding fragments of the G-CSF protein, albumin proteins, and/or albumin fusion proteins can also be used. Even if deletion of one or more amino acids of a protein results in modification or loss of one or more biological functions of the G-CSF protein, albumin protein, and/or albumin fusion protein, other therapeutic activities and/or functional activities (e.g., biological activities, ability to multimerize, ability to bind a ligand) can still be retained. For example, the ability of polypeptides with deletions to induce and/or bind to antibodies which recognize the complete or mature forms of the polypeptides generally will be retained when less than the majority of the residues of the complete polypeptide are removed.

N-terminal deletions can be described by the general formula m to q, where q is a whole integer representing the total number of amino acid residues in a reference polypeptide (e.g., a G-CSF protein, albumin protein, or albumin-G-CSF fusion protein), and m is defined as any integer ranging from 2 to q minus 6. Polynucleotides encoding these polypeptides are also provided.

C-terminal deletions can be described by the general formula I to n, where n is any whole integer ranging from 6 to q minus 1, and where q is a whole integer representing the total number of amino acid residues in a reference polypeptide (e.g., a G-CSF protein, an albumin protein, or a G-CSF-albumin fusion protein). Polynucleotides encoding these polypeptides are also provided. The present application is also directed to proteins containing polypeptides at least about 80%, about 85%, about 90%, about 91%, about 92%, about 93%, about 94%, about 95%, about 96%, about 97%, about 98% or about 99% identical to a reference G-CSF polypeptide or a reference albumin polypeptide set forth herein, or fragments thereof, e.g., a polypeptide shown in FIG. 1 (SEQ ID NO:2). In some embodiments, the G-CSF or albumin polypeptide is identical to a reference G-CSF or albumin polypeptide (e.g., a polypeptide shown in FIG. 1; SEQ ID NO:2) except for no more than 15, 14, 13, 12, 10, 9, 8, 7, 6, 5, 4, 3, 2, or 1 amino acids. In some embodiments, the application is directed to proteins comprising polypeptides at least about 80%, about 85%, about 90%, about 91%, about 92%, about 93%, about 94%, about 95%, about 96%, about 97%, about 98% or about 99% identical to reference polypeptides having the amino acid sequence of N- and C-terminal deletions as described above. Polynucleotides encoding these polypeptides are also encompassed by the invention.

Polypeptide fragments of the invention include fragments comprising, or alternatively, consisting of, an amino acid sequence that displays a therapeutic activity and/or functional activity (e.g. biological activity) of the polypeptide sequence of the G-CSF protein or serum albumin protein of which the amino acid sequence is a fragment. Other polypeptide fragments are biologically active fragments. Biologically active fragments are those exhibiting activity similar, but not necessarily identical, to an activity of the polypeptide of the present invention. The biological activity of the fragments can include an improved desired activity, or a decreased undesirable activity.

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant”, refers to a G-CSF protein portion of an albumin fusion protein, albumin portion of an albumin fusion protein, or albumin fusion protein differing in sequence from a G-CSF protein, albumin protein, and/or albumin fusion protein, respectively, but retaining at least one functional and/or therapeutic property thereof as described elsewhere herein or otherwise known in the art. Generally, variants are overall very similar, and, in many regions, identical to the amino acid sequence of the G-CSF protein corresponding to a G-CSF protein portion of an albumin fusion protein, albumin protein corresponding to an albumin protein portion of an albumin fusion protein, and/or albumin fusion protein. Nucleic acids encoding these variants are also encompassed by the invention.

Further polypeptides encompassed by the invention are polypeptides encoded by polynucleotides which hybridize to the complement of a nucleic acid molecule encoding an albumin fusion protein of the invention under stringent hybridization conditions (e.g., hybridization to filter bound DNA in 6×. Sodium chloride/Sodium citrate (SSC) at about 45 degrees Celsius, followed by one or more washes in 0.2×SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95%-“identical” to a query amino acid sequence, it is intended that the amino acid sequence of the subject polypeptide is identical to the query sequence except that the subject polypeptide sequence can include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence can occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, about 85%, about 90%, about 91%, about 92%, about 93%, about 94%, about 95%, about 96%, about 97%, about 98% or about 99% identical to, for instance, the amino acid sequence of an albumin fusion protein or a fragment thereof (such as a G-CSF protein portion of the albumin fusion protein or an albumin portion of the albumin fusion protein), can be determined conventionally using known computer programs. One method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of the global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made.

Homology or identity at the nucleotide or amino acid sequence level is determined by BLAST (Basic Local Alignment Search Tool) analysis using the algorithm employed by the programs blastp, blastn, blastx, tblastn and tblastx (Karlin et al., Proc. Natl. Acad. Sci. USA 87: 2264-2268 (1990) and Altschul, J. Mol. Evol. 36: 290-300 (1993), fully incorporated by reference) which are tailored for sequence similarity searching.

The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters may be adjusted as follows: Q-10 (gap creation penalty); R=10 (gap extension penalty); wink=1 (generates word hits at every wink.sup.th position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The polynucleotide variants of the invention can contain alterations in the coding regions, non-coding regions, or both. In some embodiments, polynucleotide variants containing alterations which produce silent substitutions, additions, or deletions, but do not alter the properties or activities of the encoded polypeptide. In some embodiments, nucleotide variants are produced by silent substitutions due to the degeneracy of the genetic code. In some embodiments, polypeptide variants contain less than 50, less than 40, less than 30, less than 20, less than 10, or 5-50, 5-25, 5-10, 1-5, or 1-2 amino acids that are substituted, deleted, or added in any combination. Polynucleotide variants can be produced for a variety of reasons, e.g., to optimize codon expression for a particular host (change codons in the human mRNA to those preferred by a bacterial host, such as, yeast or E. coli).

For example, a polynucleotide that encodes the albumin and/or G CSF portion of an albumin fusion protein can be optimized for expression in yeast or mammalian cells.

In an alternative embodiment, a codon optimized polynucleotide which encodes a G-CSF protein, an albumin protein, or an albumin-GCSF fusion does not hybridize to the wild type polynucleotide encoding the G-CSF, albumin, or fusion protein under stringent hybridization conditions as described herein

In an additional embodiment, a polynucleotide which encodes a G-CSF protein, albumin protein, or albumin-GCSF fusion does not comprise, or alternatively consist of, the naturally occurring sequence of that G-CSF, albumin, or fusion protein.

Using known methods of protein engineering and recombinant DNA technology, variants can be generated to improve or alter the characteristics of albumin-G-CSF fusions. For instance, one or more amino acids can be deleted from the N-terminus or C-terminus of the polypeptides without substantial loss of biological function.

In some embodiments, the variants of the albumin-G-CSF proteins have conservative substitutions. By “conservative substitutions” is intended swaps within groups such as replacement of the aliphatic or hydrophobic amino acids Ala, Val, Leu and Ile; replacement of the hydroxyl residues Ser and Tar; replacement of the acidic residues Asp and Glu; replacement of the amide residues Asn and Gln, replacement of the basic residues Lys, Arg, and H is; replacement of the aromatic residues Phe, Tyr, and Trp, and replacement of the small-sized amino acids Ala, Ser, Thr, Met, and Gly.

Guidance concerning how to make phenotypically silent amino acid substitutions is provided, for example, in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. See Cunningham and Wells, Science 244:1081-1085 (1989). The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys. Arg, and H is; replacement of the aromatic residues Phe, Tyr, and Trp, and replacement of the small-sized amino acids Ala, Ser, Thr, Met, and Gly. Besides conservative amino acid substitution, variants of the present invention include (i) polypeptides containing substitutions of one or more of the non-conserved amino acid residues, where the substituted amino acid residues may or may not be one encoded by the genetic code, or (ii) polypeptides containing substitutions of one or more of the amino acid residues having a substituent group, or (iii) polypeptides which have been fused with or chemically conjugated to another compound, such as a compound to increase the stability and/or solubility of the polypeptide (for example, polyethylene glycol), (iv) polypeptide containing additional amino acids, such as, for example, an IgG Fc fusion region peptide. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. See Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).

In specific embodiments, the albumin-G-CSF polypeptides comprise, consist essentially of, or, consist of, fragments or variants of the amino acid sequence of an albumin fusion protein, the amino acid sequence of a G-CSF protein and/or human serum albumin, wherein the fragments or variants have 1-5, 5-10, 5-25, 5-50, 10-50 or 50-150, amino acid residue additions, substitutions, and/or deletions when compared to the reference amino acid sequence. In some embodiments, the amino acid substitutions are conservative. Nucleic acids encoding these polypeptides are also provided.

The albumin-G-CSF fusions can be composed of amino acids joined to each other by peptide bonds or modified peptide bonds, i.e., peptide isosteres, and can contain amino acids other than the 20 gene-encoded amino acids. The polypeptides can be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:4862 (1992)).

### V. FUNCTIONAL ACTIVITY

“A polypeptide having functional activity” refers to a polypeptide capable of displaying one or more known functional activities associated with the full-length, pro-protein, and/or mature form of a G-CSF, albumin, or albumin-G-CSF fusion protein. Such functional activities include, but are not limited to, biological activity, antigenicity [ability to bind (or compete with a polypeptide for binding) to an anti-polypeptide antibody], immunogenicity (ability to generate antibody which binds to a specific polypeptide of the invention), ability to form multimers with polypeptides of the invention, and ability to bind to a receptor or ligand for a polypeptide.

“A polypeptide having biological activity” refers to a polypeptide exhibiting activity similar to, but not necessarily identical to, an activity of a G-CSF, albumin, or albumin-G-CSF fusion protein, including mature forms, as measured in a particular biological assay, with or without dose dependency.

In some embodiments, an albumin fusion protein of the invention has at least one biological and/or therapeutic activity associated with the G-CSF protein portion (or fragment or variant thereof) when it is not fused to albumin.

The albumin fusion proteins of the invention can be assayed for functional activity (e.g., biological activity) using or routinely modifying assays known in the art, as well as assays described herein. Additionally, one of skill in the art can routinely assay fragments of a G-CSF protein corresponding to a G-CSF protein portion of an albumin fusion protein. Further, one of skill in the art can routinely assay fragments of an albumin protein corresponding to an albumin protein portion of an albumin fusion protein, for activity using assays known in the art and/or as described herein. For instance, functional assays for G-CSF, albumin, and G-CSF albumin fusion proteins are described in International Publication Nos. WO 2010/083434 and WO 2010/083439, each of which is incorporated by reference herein in its entirety.

### VI. FUSION PROTEINS OF G-CSF AND HSA

Examples of recombinant human albumin-human granulocyte colony stimulating factors (rHA-G-CSF) are included in U.S. Pat. No. 5,665,863 and in U.S. Pat. No. 7,041,478, both of which are hereby incorporated by reference.

Another example of rHA-G-CSF is Neugranin™ (“NEUG”) developed by Teva Biopharmaceuticals USA LTD. NEUG is a fusion polypeptide with a molecular mass of approximately 85 kDa. NEUG is a 759 amino acid single chain polypeptide, with residues 1-585 corresponding to the mature form of HSA, and residues 586-759 corresponding to the mature form of human G-CSF. The amino acid sequence of the NEUG fusion protein is shown in FIG. 1.

In some embodiments, the albumin is N-terminal to the G-CSF protein. In other embodiments, the G-CSF is N-terminal to the albumin protein. In some embodiments, the albumin and G-CSF protein sequences are directly fused. In some embodiments, the albumin and G-CSF protein sequences are fused indirectly, e.g., via a linker sequence.

### VI. PRODUCING THE FUSION PROTEIN

Exemplary methods of synthetic processes of manufacture of rHA-G-CSF are described in U.S. Patent Published Application No. 2008/0153751 hereby incorporated by reference in its entirety. In some embodiments, a G-CSF protein or albumin-G-CSF fusion such as NEUG is produced using a yeast host system (e.g., Saccharomyces cerevisiae) genetically engineered to express the albumin-G-CSF fusion, e.g., NEUG fusion, protein. The albumin-G-CSF, e.g., NEUG, can be harvested from the fermentation medium of the yeast culture and purified using methods well known in the art (e.g., by a series of chromatography and filtration steps, such as affinity chromatography and ion exchange chromatography).

In one non-limiting example, a NEUG fusion construct was developed as follows. The full-length albumin cDNA was isolated from a human cDNA library in the laboratory of Dr. F. E. Baralle at the University of Oxford, UK. This clone was sent to Delta Biotechnology Limited, Nottingham, UK, as the plasmid pAT153ALB. In addition, the 6-amino acid HSA pro-peptide (RGVFRR) was modified to facilitate more efficient processing in yeast (RSLDKR).

The NEUG production plasmid, a modified pSAC35-based expression vector, is based on the 2-μplasmid found in wild type Saccharomyces cerevisiae. The pSAC35-based expression vector (see e.g., patents EP 286 424 B, U.S. Pat. No. 5,637,504) contains the LEU2 gene from Saccharomyces cerevisiae as a selectable marker that complements the leucine-deficiency of the S. cerevisiae production host. This production plasmid also contains a strong yeast promoter, PRB1, and sequences from plasmid pUC9 that permit cloning and propagation in E. coli. In addition, the plasmid eliminates the pUC9-derived sequences required for propagation in E. coli once transformed into yeast. This is accomplished by flanking FLP recognition targets (FRT) and the expression of the yeast FLP recombinase from the plasmid once in yeast. Thus, no bacterial DNA is present in the organism used for production of NEUG. This is confirmed by rescue and sequence of the 2 μm plasmid from the yeast after the master cell bank is generated.

As described above, the NEUG production plasmid, termed CID1643 (pSAC35:HSA.GCSF(T31-P204)), was derived from the pSAC35-based expression vector. The region corresponding to T31-P204 of human G-CSF was amplified by PCR, while adding the appropriate 5′ and 3′ restriction sites to permit a seamless fusion to the 3′-end of the HSA open reading frame.

NEUG seed vials were used to prepare a cGMP master cell bank at Human Genome Sciences, Inc., in Rockville, Md. The testing and characterization of the NEUG master cell bank was undertaken at Charles River Laboratories (Malvern, Pa., USA) and Lark Technologies (Houston, Tex., USA) in compliance with the ICH guideline Q5D (Derivation and Characterization of Cell Substrates Used for Production of Biotechnological/Biologicals Products).

A cGMP working cell bank derived from this master cell bank was subsequently generated and tested at Charles River Laboratories (Malvern, Pa., USA).

All media components used in the manufacture of the NEUG cell line banks were synthetic, biosynthetic or plant derived. No components of animal or human origin were used during cell line or cell bank preparation.

The cell banks is stored at <−135° C. in a cryopreservation media in pre-sterilized 1.8 mL Nunc polypropylene tubes with internally threaded caps.

The formulated drug substance is aseptically filtered using a 0.2 μm filter into autoclaved Teflon bottles. The liquid filled drug substance is stored frozen at about −80° C. (nominal value, acceptable range of storage temperature is about −65° C.).

To improve the robustness of the formulation for shipping and storage at clinical sites as well as to provide a stable product with an expected long shelf life, NEUG may also be lyophilized by methods well known in the art.

Albumin-G-CSF fusion proteins can also be expressed and purified from any other known recombinant protein expression known in the art. These include but are not limited to microorganisms such as bacteria (e.g., E. coli, B. subtilis) transformed with recombinant bacteriophage DNA, plasmid DNA or cosmid DNA expression vectors containing albumin-G-CSF coding sequences; yeast (e.g., Saccharomyces, Pichia) transformed with recombinant yeast expression vectors containing albumin-G-CSF coding sequences; insect cell systems infected with recombinant virus expression vectors (e.g., baculovirus) containing albumin-G-CSF coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing albumin-G-CSF coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or eukaryotic cells, can be used for the expression of a recombinant albumin-G-CSF molecule. In addition, mammalian cells such as Chinese hamster ovary cells (CHO), in conjunction with a vector such as the major intermediate early gene promoter element from human cytomegalovirus is an effective expression system for albumin-G-CSF.

### VII. EXEMPLARY CAUSES OF LEUKOPENIA AND NEUTROPENIA

As described above, leukopenia is a reduction in the circulating white blood cells (WBC) count, and neutropenia is characterized by a reduction in the blood neutrophil count, often leading to increased susceptibility to bacterial and fungal infections. The following is a non-comprehensive list of factors that can place a human subject at risk of developing leukopenia or neutropenia: drugs (e.g. phenyloin, chloramphenicol, sulfa drugs, and chemotherapy); vitamin B12 or folate deficiency; excessive alcohol consumption; cancer or other diseases which involve the bone marrow (e.g. aplastic anemia, dysgammaglobulinemia, paroxysmal nocturnal hemoglobinemia, myelodysplasia, myelodysplastic syndromes, myelofibrosis, leukemia, myeloma, lymphoma, or metastatic solid tumors which infiltrate and replace the bone marrow); viral infections (e.g. influenza, HIV, early-stage infectious mononucleosis, childhood viral diseases); bacterial infections (e.g. tuberculosis); radiation; toxins (e.g., benzene and insecticides); bone marrow failure (e.g. Schwachman-Diamond syndrome, cartilage-hair hypoplasia, dyskeratosis congenita, glycogen storage disease type IB); spleen disorder, splenomegaly of any cause; intrinsic defects in myeloid cells or their precursors; allergic disorders; autoimmune disease; T-γ lymphoproliferative disease (T-γ LPD); hemodialysis or transplantation; and toxins.

Numerous drugs, such as many chemotherapy regimens (e.g., cytotoxic chemotherapy regimens), are associated with a high risk of febrile neutropenia (e.g., >than 20% risk). In some chemotherapy regimens, the incidence of febrile neutropenia in the absence of G-CSF treatment is about 40% (e.g., a chemotherapy regimen of intravenous doxorubicin and docetaxel). Non-limiting examples of various cancers and treatment regimens associated with febrile neutropenia risk are provided below in Table 1. In some embodiments, the HSA-G-CSF fusion protein of FIG. 1 is administered to a patient to prevent, treat or ameliorate neutropenia associated with the administration of such drug therapies.

TABLE 1
Exemplary cancers and treatment regimens associated with febrile neutropenia

| Cancer | Treatment |
|---|---|
| Bladder Cancer | MVAC (methotrexate, vinblastine, doxorubicin, cisplatin) |
| | (neoadjuvant, adjuvant, metastatic) |
| Breast Cancer | Docetaxel + trastuzumab (metastatic or relapsed) |
| | Dose dense AC-T (doxorubicin, cyclophosphamide, |
| | paclitaxel) (adjuvant) |
| | AT (doxorubicin, paclitaxel) (metastatic or relapsed) |
| | AT (doxorubicin, docetaxel) (metastatic or relapsed) |
| | TAC (docetaxel, doxorubicin, cyclophosphamide) (adjuvant) |
| Esophageal and Gastric Cancer | Docetaxel/cisplatin/fluorouracil |
| Non-Hodgkin's Lymphoma | ICE (ifosfamide, carboplatin, etoposide) (Diffuse Large B- |
| | Cell Lymphoma, Peripheral T cell Lymphomas, 2nd line, |
| | salvage) |
| | RICE (rituximab, ifosfamide, carboplatin, etoposide) |
| | CHOP-14 (cyclophosphamide, doxorubicin, vincristine, |
| | prednisone) |
| | MINE (mesna, ifosfamide, novantrone and etoposide) |
| | (Diffuse Large B-Cell Lymphoma, Peripheral T cell |
| | Lymphomas, 2nd line, refractory) |
| | DHAP (dexamethasone, cisplatin, cytarabine) (Peripheral T |
| | cell Lymphomas, Diffuse Large B-Cell Lymphoma, 2nd line) |
| | ESHAP (etoposide, methylprednisolone, cisplatin, |
| | cytarabine) (Diffuse Large B-Cell Lymphoma, Peripheral T |
| | cell Lymphoma, 2nd line, recurrent) |
| | BEACOPP (bleomycin, etoposide, doxorubicin, |
| | cyclophosphamide, vincristine, procarbazine, prednisone) |
| | HyperCVAD + Rituximab (cyclophosphamide, vincristine, |
| | doxorubicin, dexamethasone + rituximab) (Burkitt's |
| | Lymphoma) |
| Melanoma | Dacarbazine-based combination (dacarbazine, cisplatin, |
| | vinblastine) (advanced, metastatic, or recurrent) |
| | Dacarbazine-based combination with IL-2, interferon alfa |
| | (dacarbazine, cisplatin, vinblastine, IL-2, interferon alfa) |
| | (advanced, metastatic, or recurrent) |
| Myelodysplastic syndrome | Decitabine |
| Ovarian Cancer | Topotecan |
| | Paclitaxel |
| | Docetaxel |
| Pancreatic Cancer | Gemcitabine/docetaxel |
| Sarcoma | MAID (mesna, doxorubicin, ifosfamide, dacarbazine) |
| | Doxorubicin |
| Small Cell Lung Cancer | Topotecan |
| Testicular Cancer | VeIP (vinblastine, ifosfamide, cisplatin) |
| | VIP (etoposide, ifosfamide, cisplatin) |
| | BEP (bleomycin, etoposide, cisplatin) |
| | TIP (paclitaxel, ifosfamide, cisplatin) |

Non-limiting examples of treatment regimens associated with febrile neutropenia risk following treatments in e.g, solid tumors in pediatric patients are provided below in Table 2.

TABLE 2
Allowed Chemotherapy Regimens for Patients
Enrolled in the Study

| Chemotherapy | |
|---|---|
| Regimen | Details of chemotherapy regimen |
| VIDE | Vincristine: 1.5 mg/m² on Day 1 |
| | Ifosfamide: 3.0 g/m² on Day 1, Day 2, and Day 3 |
| | Doxorubicin: 20 mg/m² on Day 1, Day 2, and Day 3 |
| | Etoposide: 150 mg/m² on Day 1, Day 2, and Day 3 |
| VDC/IE | VDC Cycles 1 and 3 |
| | Vincristine: 2.0 mg/m² on Day 1, Day 8, and Day 15 |
| | Doxorubicin: 75 mg/m² on Day 1 |
| | Cyclophosphamide: 1200 mg/m² on Day 1 and Day 2 |
| | IE Cycles 2 and 4 |
| | Ifosfamide: 1800 mg/m²/day for 5 days (CTX Days 1-5_{—} |
| | Etoposide: 100 mg/m²/day for 5 days (CTX Days 1-5) |
| IVA and IVAd | IVA Cycles 1, 2 and 4 |
| | Ifosfamide: 3.0 g/m² on CTX Day 1 and CTX Day 2 |
| | Vincristine: 1.5 mg/m² on CTX Day 1, CTX Day 8, and |
| | CTX Day 15 |
| | Actinomycin D 1.5 mg/m² on CTX Day 1 |
| | IVA Cycle 3 |
| | Ifosfamide: 3.0 g/m² on CTX Day 1 and CTX Day 2 |
| | Vincristine: 1.5 mg/m² on CTX Day 1 |
| | Actinomycin D 1.5 mg/m² on CTX Day 1 |
| | IVAd Cycles 1 and 4 |
| | Ifosfamide: 3.0 g/m² on CTX Day 1 and CTX Day 2 |
| | Vincristine: 1.5 mg/m² on CTX Day 1, CTX Day 8, and |
| | CTX Day 15 |
| | Adriamycin (Doxorubicin) 20 mg/m² twice daily on |
| | CTX Day 1 and CTX Day 2 |
| | IVAd Cycle 2 |
| | Ifosfamide: 3.0 g/m² on CTX Day 1 and CTX Day 2 |
| | Vincristine: 1.5 mg/m² on CTX Day 1, CTX Day 8, and |
| | CTX Day 15 |
| | Actinomycin D 1.5 mg/m² on CTX Day 1 |
| | IVAd Cycle 3 |
| | Ifosfamide: 3.0 g/m² on CTX Day 1 and CTX Day 2 |
| | Vincristine: 1.5 mg/m² on CTX Day 1 |
| | Adriamycin (Doxorubicin) 20 mg/m² twice daily on |
| | CTX Day 1 and CTX Day 2 |

CTX = chemotherapy;
IE = Ifosfamide plus etoposide;
IVA = Ifosfamide plus vincristine plus actinomycin D;
IVAd = Ifosfamide plus vincristine plus actinomycin D/adriamycin (doxorubicin twice daily);
VIDE = Vincristine plus ifosfamide plus doxorubicin plus etoposide;
VDC = Vincristine plus doxorubicin plus cyclophosphamide.

Cytotoxic treatment regimens for small cell lung carcinoma e.g., cisplatin plus etoposide, as well as CAE, are also associated with febrile neutropenia.

Various neutropenias are known, and in some embodiments, the HSA-G-CSF fusion protein of FIG. 1 is used to prevent, treat or ameliorate one or more neutropenias, including, but not limited to chemotherapy induced neutropenia, febrile neutropenia, primary neutropenia, acute neutropenia, severe chronic neutropenia (SCN), severe congenital neutropenia (Kostmann's syndrome), severe infantile genetic agranulocytosis, benign neutropenia, cyclic neutropenia, chronic idiopathic neutropenia, secondary neutropenia, syndrome associated neutropenia, or immune-mediated neutropenia.

### VIII. EXPERIMENTAL EXAMPLES

The following examples are given to illustrate the present invention. It should be understood, however, that the invention is not to be limited to the specific conditions or details described in these examples. All publically available documents referenced herein, including but not limited to US patents are hereby incorporated by reference.

### Study Product and Production

Balugrastim is a contiguous 759 amino acid, fully recombinant protein composed of the mature form of human serum albumin (HSA) seamlessly joined at its carboxy terminus to the amino terminus of human granulocyte-colony stimulating factor (hG-CSF). Balugrastim is expressed in the yeast Saccharomyces cerevisiae, without mutations or linkers in either component or the junction between them, resulting in an 85 kD protein.

The hG CSF component of Balugrastim is fully active and regulates the proliferation and differentiation of progenitor cells within the bone marrow and release of mature neutrophils into the peripheral bloodstream. The human HSA component of Balugrastim acts as a carrier of G CSF, extending its plasma half life by minimizing renal clearance. The main elimination of Balugrastim is via receptor mediated neutrophil endocytosis.

The proposed indication for Balugrastim is the reduction in the duration of neutropenia and the incidence of febrile neutropenia in patients treated with cytotoxic chemotherapy for malignancy (with the exception of chronic myeloid leukemia and myelodysplastic syndromes).

Presumably, a long-acting G-CSF would decrease the incidence of infection, as manifested by febrile neutropenia, in a human subject with non-myeloid malignancies that is less than 18 years old.

### Formulation

In Example 1 below, Balugrastim drug product is supplied in a lyophilized form, 25 mg, sterile, lyophilized formulation (freeze dried product) in single use, 3 mL type 1 glass vials, which are sealed with a coated rubber stopper and a flip off seal. Balugrastim for injection should be stored at 2 to 8° C. Upon reconstitution with 0.6 mL of sterile water for injection (SWFI), each vial contains 50 mg/mL (25 mg/vial deliverable) Balugrastim in 20 mM sodium phosphate, 180 mM mannitol, 60 mM trehalose dihydrate, 0.06% (w/v) polysorbate 80 at pH 6.0. After reconstitution the vial can be held at room temperature and should be used within 8 hours.

### Dosage Selection

The dosages that are being used in this study (300 μg/kg and 670 μg/kg approximately 24 hours after chemotherapy for up to 4 cycles) are selected based on 2 safety/PK studies in adults (study NEUGR 01 and study NEUGR 02).

The dosages of 300 μg/kg and 670 μg/kg for this study are selected based on the current safety and efficacy data available for adults treated with Balugrastim with doses up to 1000 μg/kg, and from published results in children of comparable doses of pegfilgrastim.

The Balugrastim dose of 300 μg/kg was already used in 23 adult patients; it was one of the treatment arms in the breast cancer study NEUGR 001. In the first part of this study, Balugrastim was administered at doses of 50, 150, 300, or 450 μg/kg to 3, 3, 4, and 3 patients, respectively. In the second part of the study, a dose of 300 or 450 μg/kg of Balugrastim, or 6 mg pegfilgrastim was administered to 20, 21, and 10 patients, respectively. In the first part of the study, during Cycle 1, there were 2 cases of grade 4 neutropenia in the 150 μg/kg, 300 μg/kg and 450 μg/kg Balugrastim treatment arms. There was also one case of grade 4 persistent neutropenia in the 450 μg/kg Balugrastim treatment arm. In the second part of the study, during Cycle 1, there were 9 cases of grade 4 neutropenia in the 300 μg/kg Balugrastim treatment arm, 6 cases in the 450 μg/kg Balugrastim treatment arm, and 3 cases in the pegfilgrastim treatment arm. There was also 1 case of grade 4 persistent neutropenia in the 450 μg/kg Balugrastim treatment arm. The mean DSN in Cycle 1 of the second part was 1.1 days for the 300 μg/kg Balugrastim treatment arm, 1.0 day for the 450 μg/kg Balugrastim treatment arm, and 0.7 day for the pegfilgrastim treatment arm respectively.

In study NEUGR 001 it was shown that Balugrastim at a dose of 300 μg/kg is safe for adult patients, but not as efficacious as the higher Balugrastim or pegfilgrastim doses. The Balugrastim dose of 670 μg/kg is equivalent to the adult fixed dose of 40 mg Balugrastim for a patient with a body weight of 60 kg. A dose of 40 mg Balugrastim has shown to have efficacy comparable to 6 mg pegfilgrastim in the pivotal breast cancer studies NEUGR 002 and NEUGR 003 that were done in adult patients.

Filgrastim was chosen as a comparator in this study because it is the only product approved for the pediatric treatment of febrile neutropenia. 5 μg/kg filgrastim will be administered subcutaneously once a day for a minimum of 5 consecutive days and a maximum of 14 days; or until the ANC returns to ≧2.0×109/L

### Example 1

### Phase II Pediatric Clinical Trial

### Purpose of the Study and Study Objectives

### Purpose of the Study

This study aims to evaluate the pharmacology, efficacy and safety of Balugrastim at dosages of 300 μg/kg and 670 μg/kg in pediatric patients diagnosed with solid tumors who are receiving chemotherapy.

### Study Objectives

Primary Objective: The primary objective of this study is to find the optimal dose of Balugrastim by characterizing its pharmacokinetics, and by comparing the pharmacodynamics of Balugrastim to filgrastim in children receiving chemotherapy.

Secondary Objective: The secondary objectives of this study are:
To document duration of severe neutropenia and the incidence of febrile neutropenia in Cycle 1 of chemotherapy; and
To assess safety, tolerability and immunogenicity of Balugrastim.

### Study Design

### General Design and Study Schema

This Phase 2 study assesses the pharmacology, efficacy and safety of a single SC dose (300 μg/kg or 670 μg/kg) of Balugrastim vs. 5 μg/kg filgrastim in 36 patients aged 2 to 17 years. Each treatment group is formed by 12 patients, six of 2 to 11 years, and six of 12 to 17 years. The study population includes patients diagnosed with solid tumors who are receiving chemotherapy. Patients are treated as inpatients or outpatients and filgrastim is used as a comparator in this study.

The chemotherapy regimens include VIDE, VDC/IE, IVA, or IVAd for patients with solid tumors. In the absence of any prophylactic WBC growth factor, WBC and neutrophil counts are expected to decrease significantly with all these regimens. Therefore, there is a clinical need for G CSFs to be administered as primary prophylaxis in pediatric patients with solid tumors who are receiving these dose-intensive chemotherapy regimens. In particular, there is a need for a prolonged acting G CSF such as Balugrastim, that is administered once per chemotherapy cycle.

Table 2 above summarizes the chemotherapy regimens that will be used in this study.

The inclusion criteria in this study include a minimum body weight of 10 kg (alternatively 15 kg) to ensure an appropriate relationship of total blood volume drawn to total blood volume needed for the PK analysis. Safety is evaluated by assessment of AEs, vital signs, clinical laboratory parameters, ECG, physical examination, concomitant medications, and immunogenicity (up to 365 days).

### General Design and Study Schema

This is a Phase 2, multicenter, open-label, randomized, active-controlled, dose-finding study to evaluate the pharmacology, efficacy and safety of balugrastim at dosages of 300 μg/kg and 670 μg/kg in pediatric patients diagnosed with solid tumors who are receiving myelosuppressive chemotherapy.

Patients, parents, or legal representatives should provide informed consent before any study-specific procedure could be initiated. Eligibility will be assessed during a screening period, estimated to last up to 14 days.

Two groups stratified by age are enrolled: 2 to 11 years and 12 to 17 years. Recruitment of the youngest cohort of patients will begin only after results of the PD (central laboratory results of ANC values) and safety data of at least 8 patients (including results from at least 4 children from the 670 μg/kg dose group) from the oldest cohort are available. The study is conducted in approximately 20 sites in 8 countries (Bulgaria, Hungary, Russia, Ukraine, Romania, Georgia, Czech Republic and Slovakia).

Balugrastim is administered SC approximately 24 (±3 h) after the end of the last chemotherapy administration in Week 1 of the specific regimen (i.e., VDC/IE regimen: Day 3 during Cycles 1 and 3, and Day 6 during Cycles 2 and 4; VIDE regimen: Day 4; IVA and IVAd regimen: Day 3), for up to 4 cycles.

The study includes a screening period of 2 weeks, a treatment period of 12 weeks (4 cycles, 84 days), an end of treatment visit to be performed 4 weeks after the end of the start of the last chemotherapy treatment (alternatively, after the last chemotherapy treatment), and a follow-up period of up to 12 months from the start of chemotherapy administration. Follow-up visits will be performed on Days 180 and 365 with relation to the start of chemotherapy treatment.

Samples for balugrastim PK assessments are taken in relation to the day of balugrastim administration according to the different chemotherapeutic regimens (Table 3). The pre-dose sample is taken within 1 hour prior to the first injection of balugrastim on Day 1 followed by sampling at 6, 12, and 24 hours post-injection. Subsequent samples are taken on Days 1, 3, 5, 7, and 9 in Cycle 1 after balugrastim administration in relation to the corresponding chemotherapy regimen (ie VIDE, VDC/IE, IVA, IVAd).

TABLE 3
Pharmacokinetics Assessments

| | | | | Study Day |
|---|---|---|---|---|
| | Time after | Study Day | Study Day | in Relation |
| | balugrastim | in Relation | in Relation | to IVA and |
| PK | administration | to VIDE^{a} | to VDC/IE^{b} | IVAd^{c} |
| sample | (h) | regimen | regimen | regimen |
| 1 | Pre-dose | 4 | 3 | 3 |
| 3 | 6 h | 4 | 3 | 3 |
| 4 | 12 h | 4 | 3 | 3 |
| 5 | 24 h (1 day) | 5 | 4 | 4 |
| 7 | 72 h (3 days) | 7 | 6 | 6 |
| 8 | 120 h (5 days) | 9 | 8 | 8 |
| 9 | 168 h (7 days) | 11 | 10 | 10 |
| 10 | 216 h (9 days) | 13 | 12 | 12 |

^{a}Vincristine plus ifosfamide plus doxorubicin plus etoposide
^{b}Vincristine plus doxorubicin plus cyclophosphamide alternating with ifosfamide plus etoposide
^{c}IVA = Ifosfamide plus vincristine plus actinomycin D; IVAd = Ifosfamide plus vincristine plus Adriamycin (doxorubicin) twice daily

Samples for PD assessments are obtained 1 hour prior to study drug administration during Cycles 1-4 on Day 1 (VDC/IE regimen: Day 3 during Cycles 1 and 3, and Day 6 during Cycles 2 and 4; VIDE regimen: Day 4; IVA and IVAd regimen: Day 3), and then at least every other day starting on Day 2 to Day 12 following drug administration (VDC/IE regimen: Days 4 to 14 during Cycles 1 and 3, and Day 7 to 17 during Cycles 2 and 4; VIDE regimen: Days 5 to 15; IVA and IVAd regimen: Days 4 to 14) until ANC≧2.0×10⁹/L after nadir, and then twice weekly during the respective cycle or the EOS (see Tables 4a and b).

TABLE 4a
Pharmacokinetics Assessments

| | PK^{a} assessments (Cycle 1) | | | | | | | |
|---|---|---|---|---|---|---|---|---|
| | (Hours post-injection) | | | | | | | |
| | −1 | | | | | | | 216 |
| | (pre- | | | 24 | 72 | 120 | 168 | (9 |
| Procedure | dose) | 6 | 12 | (1 day) | (3 days) | (5 days) | (7 days) | days) |
| PK | X | X | X | X | X | X | X | X |
| PD^{b} | | | | | | | | |

TABLE 4B
Pharmacodynamics Assessments

| | PD Assessments^{b} (Cycles 1-4) | | | | |
|---|---|---|---|---|---|
| | −1 | | Day 2 | D4-D12 | |
| | (pre- | | following | following | |
| Procedure | dose) | D1 | SD admin. | SD admin. | EOS |
| PK | | | | | |
| PD^{c} | X | X | X | Every other | X |
| | | | | day | |

^{a}Balugrastim will be administered SC approximately 24 hours (±3) after the end of the last chemotherapy administration in Week 1 of the specific regimen (VDC/IE regimen: chemotherapy Day 3 during Cycles 1 and 3, and on chemotherapy Day 6 during Cycles 2 and 4; VIDE regimen: chemotherapy Day 4; IVA and IVAd regimen: chemotherapy Day 3), for up to 4 cycles.
^{b}Balugrastim will be administered SC approximately 24 hours (±3) after the end of the last chemotherapy administration in Week 1 of the specific regimen (VDC/IE regimen: chemotherapy Day 3 during Cycles 1 and 3, and on chemotherapy Day 6 during Cycles 2 and 4; VIDE regimen: chemotherapy Day 4; IVA and IVAd regimen: chemotherapy Day 3), for up to 4 cycles.
^{c}Samples for PD assessments will be obtained 1 hour prior to study drug administration during Cycles 1-4 on Day 1 (VDC/IE regimen: Day 3 during Cycles 1 and 3, and Day 6 during Cycles 2 and 4; VIDE regimen: Day 4; IVA and IVAd regimen: Day 3), and then at least every other day starting on Day 2 to Day 12 following drug administration (VDC/IE regimen: Days 4 to 14 during Cycles 1 and 3, and Days 7 to 17 during Cycles 2 and 4; VIDE regimen: Days 5 to 15; IVA and IVAd regimen: Days 4 to 14) until ANC ≧2 × 10⁹/L after nadir, and then twice weekly during the respective cycle or the EOS.
admin = administration;
D = day;
SD = study drug.

Safety evaluations include the incidence of AEs, vital signs including body temperature, ECG parameters, clinical laboratory parameters, spleen sonography, immunogenicity, and concomitant medications.

Samples for immunogenicity are obtained prior to the first administration of balugrastim in Cycle 1, at the end of study visit, and on follow up visits (Days 180 and 365).

### Primary and Secondary Measures and Endpoints

The primary pharmacodynamic measure for this study is:
Area under the curve of ANC (AUC_{ANC}) in Cycle 1.
The secondary pharmacodynamic variables and endpoints for this study are:
ANC nadir (measured in 10⁹/L), which is the lowest ANC recorded;
Time to ANC nadir, which is the time from the beginning of chemotherapy up to the occurrence of the ANC nadir; and
Time to ANC recovery (ANC>1.5×10⁹/L) from nadir in all treatment cycles.
The efficacy variables and endpoints for this study are:
DSN in Cycles 1-4;
Incidence of severe neutropenia in Cycles 1-4; and
Frequency of febrile neutropenia (defined as body temperature>38.5° C. for more than one hour [axillary measurement] and ANC<0.5×10⁹/L) by cycle and across all cycles.

### Secondary Pharmacokinetic Measures and Endpoints

The pharmacokinetic variables and endpoints for this study are:
Area under the curve to the time of last measurable concentration (AUC₀₋ₜₗₐₛₜ);
Area under the curve from O-time extrapolated to infinity (AUC_{0-inf});
Maximum observed serum concentration (Cₘₐₓ);
Time to maximum observed serum concentration (Tₘₐₓ);
Slope of terminal phase (λ_{z});
Terminal phase half-life (T_{1/2});
Mean residence time (MRT);
Apparent systemic clearance (CL/F); and
Apparent volume of distribution, terminal phase (V_{z}/F).

### Safety Measures and Endpoints

Secondary endpoints evaluate the safety and tolerability of a 300 μg/kg and 670 μg/kg single dose of balugrastim administered SC versus filgrastim. These include the assessment of AEs, vital signs, clinical laboratory parameters, ECG, spleen sonography, and immunogenicity. Blood samples for immunogenicity testing for balugrastim treated subjects are taken prior to the first balugrastim administration in Cycle 1, and Cycle 4 (pre-dose), at EOS and during follow up visits (approximately on Days 180 and 365). In case of antibodies signal at 12 months additional testing will be suggested if any risk or medical problem is associated with the development of ADAs. In addition, the effect of treatment on mortality due to infections and overall mortality is examined.

### Randomization and Blinding

This is an open-label, active-controlled, randomized dose-finding study. Randomization is 1:1:1. There is no blinding during the study.

### Study Drugs and Dosage

Patients receive their chemotherapy treatments as scheduled in Table 2.

### Investigational Product and Dosage

### Balugrastim

Balugrastim is a fully recombinant protein composed of HSA and G CSF. The fusion protein contains no linker sequence; the C-terminus of HSA is linked directly to the amino terminus of G-CSF via a peptide bond. Balugrastim is a sustained exposure form of human G CSF (hG CSF). Genetically combining hG CSF with HSA, a carrier protein with no intrinsic activity but a long circulation half life, extends systemic circulation of hG CSF and prolongs its therapeutic activity.

Balugrastim is produced using a yeast host system (Saccharomyces cerevisiae) genetically engineered to express the balugrastim protein. Balugrastim is harvested from the fermentation medium of the yeast culture and purified by a series of chromatography and filtration steps.

Balugrastim is a white to off-white cake in a vial.

### Formulation

Balugrastim for injection, 25 mg, is supplied as a sterile, lyophilized formulation (freeze-dried product) in single-use, 3 mL type-1 glass vials, which are sealed with a coated rubber stopper and a flip-off seal. Balugrastim for injection should be stored at 2 to 8° C. Upon reconstitution with 0.6 mL of sterile water for injection (SWFI), each vial contains 50 mg/mL (25 mg/vial deliverable) balugrastim in 20 mM sodium phosphate, 180 mM mannitol, 60 mM trehalose dihydrate, 0.06% (w/v) polysorbate 80 at pH 6.0. After reconstitution, the vial can be held at room temperature and should be used within 8 hours.

### Storage

Study drug supplies will be kept in a secure, limited-access, temperature-controlled refrigerated (2° to 8° C./36 to 46° F.) area.

### Dose, Route of Administration, and Schedule

The vials are reconstituted with 0.6 mL of water for injection to give a final concentration of 50 mg/mL and a deliverable content of 25 mg balugrastim. Reconstituted balugrastim can be stored at room temperature, but must be administered within 12 hours of reconstitution. The content of the vial (balugrastim 25 mg) is extracted via syringe with clear markings to provide an accurate dose for pediatric administration. The selected dosage, 300 μg/kg or 670 μg/kg, is administered by SC injection once per chemotherapy cycle approximately 24 h after chemotherapy administration for up to 4 cycles.

The SC injection of 300 μg/kg or 670 μg/kg balugrastim is made using a fine graded syringe and a 29G×½ inch injection needle.

### Other Study Drugs and Dosage

### Filgrastim

Filgrastim is a methionyl hG-CSF, produced by recombinant DNA technology. Filgrastim is a water-soluble 175 amino acid protein with a molecular weight of approximately 19 kD. Filgrastim is obtained from the bacterial fermentation of a strain of Escherichia coli transformed with a genetically engineered plasmid containing the hG-CSF gene.

### Formulation

Filgrastim is commercially available as a sterile, clear, colorless, preservative-free liquid for parenteral administration containing filgrastim at a specific activity of 1.0±0.6×10⁸ U/mg (as measured by a cell mitogenesis assay). The product is available in single use vials and prefilled syringes. For the purpose of this study single-use vials will be used.

Filgrastim, 300 μg/mL single use vial, manufactured by Amgen and marketed in the EU will be purchased for this study.

### Storage

Filgrastim will be stored at 2° to 8° C. (36° to 46° F.). Shaking will be avoided. Prior to injection, filgrastim may be allowed to reach room temperature for a maximum of 24 h. Any vial left at room temperature for more than 24 h will be discarded. Parenteral drug products should be inspected visually for particles and discoloration prior to administration. If particles or discoloration are observed, the product will not be used.

### Dose, Route of Administration and Schedule

Filgrastim is administered at a dose of 5 μg/kg SC once a day for at least 5 consecutive days and a maximum of 14 days, or until ANC has returned to ≧2.0×10⁹/L.

### Duration of Patient Participation

This study consists of a 2-week screening period, a 12-week treatment period (4 cycles of 21 days each), and a follow-up period of up to 12 months from the start of treatment. Follow-up visits will take place on Day 180 and Day 365 from the start of chemotherapy. Patients are expected to participate in this study for 379 days.

### Stopping Rules and Discontinuation Criteria

### Early Discontinuation

Early termination procedures are completed within 5 (±2) days after the site has been informed of a patient's decision to discontinue the study, or the Investigator has decided to withdraw a patient. If a patient is withdrawn or chooses to withdraw, the same procedures described for the end of study evaluation is offered to the patient. These include AEs assessment, vital signs, clinical laboratory tests, spleen sonography, and immunogenicity test.

AEs are reported up to 30 days after the last dose of study medication. Patients withdrawn from the study due to any AEs are followed up until the medical condition returns to baseline or is considered stable and are recorded in the patient's source documents and in the CRF.

### Criteria for Withdrawal

A patient may withdraw or be withdrawn from the study for the following reasons:
1. Patient's parent/legal representative withdrew consent or strong objections to continuing in the study from the patient
2. Sponsor requested patient to be withdrawn
3. Request of primary care physician or Investigator
4. Protocol violation/non-compliance
5. Lost to follow-up/failure to return
6. AE (specify primary AE in the CRF)
7. Lack of efficacy
8. Pregnancy
9. Death
10. A delay of more than 14 days in chemotherapy administration
11. Other

### Replacement of Withdrawn Subjects

Patients who withdraw or are withdrawn after receiving study drug are not replaced.

### Maintenance of Randomization and Blinding

This is an open-label study and no blinding is performed during the study.

### Study Procedures

The study will consist of 3 periods: Screening, Treatment and Follow up Period.

Screening period: screening to confirm eligibility must be completed within 14 days or less prior to the initiation of chemotherapy.

Treatment period: the treatment period begins at the start of chemotherapy and ends after the last PD assessment at the EOS evaluation. This period may last up to 12 weeks if chemotherapy is administered as scheduled, or a maximum of 18 weeks when allowing for delays in chemotherapy administration of up to 14 days per cycle, and includes:
up to 4 cycles of chemotherapy
balugrastim will be administered SC approximately 24 hours (±3) after the end of the last chemotherapy administration in Week 1 of the specific regimen (VDC/IE regimen: Day 3 during Cycles 1 and 3, and Day 6 during Cycles 2 and 4; VIDE regimen: Day 4; IVA and IVAd regiment: Day 3), for up to 4 cycles.
if filgrastim is used, its administration will begin not earlier than 24 hours (±3 hours) after the end of chemotherapy treatment. Filgrastim will be administered for at least 5 consecutive days in each treatment cycle.
PK assessments for balugrastim treated subjects. Samples will be taken in relation to the day of balugrastim administration according to the different chemotherapy regimen as indicated in Table 3. The pre dose sample will be taken within 1 hour prior to the first injection of balugrastim, followed by sampling at 6, 12, 24 (Day 1), 72 (Day 3), 120 (Day 5), 168 (Day 7), and 216 (Day 9) hours post injection of balugrastim in Cycle 1.
Samples for PD assessments will be obtained 1 hour prior to study drug administration during Cycles 1-4 on Day 1 (VDC/IE regimen: Day 3 during Cycles 1 and 3, and Day 6 during Cycles 2 and 4; VIDE regimen: Day 4; IVA and IVAd regimen: Day 3), and then at least every other day starting on Day 2 to Day 12 following drug administration (VDC/IE regimen: Days 4 to 14 during Cycles 1 and 3, and Day 7 to 17 during Cycles 2 and 4; VIDE regimen: Days 5 to 15; IVA and IVAd regimen: Days 4 to 14) until ANC≧2.0×10⁹/L after nadir, and then twice weekly during the respective cycle or the EOS (see Table 7).

End of cycle evaluation generally on Day 21 of the chemotherapy cycle, before the start of the next chemotherapy cycle

EOS: assessments will be performed 4 weeks after the last chemotherapy.

Follow up period: the follow up period includes 2 visits at Day 180 and at Day 365 from start of chemotherapy. Immunogenicity testing, survival, height and weight, use of G CSF, and tumor progression (Investigator's assessment) will also be documented at these visits.

The maximum duration of the study for an individual patient (from screening period until the end of the follow-up period) will be approximately 379 days (approximately 12 to 13 months).

Study procedures and assessments with their timing are summarized in Table 5 (VIDE chemotherapy regimen) and Table 6 (VDC/IE chemotherapy regimen).

TABLE 5
Schedule of Assessments - VIDE Chemotherapy Regimen

| | | | Open-label treatment period/ | | | | | | | | | |
|---|---|---|---|---|---|---|---|---|---|---|---|---|
| | Pretreatment | | (Cycles 1 through 4) (visit/week) | | | | | | | | | |
| Procedures | V1 | V2 | V3 | V4 | V5 | V6 | V7 | V8 | V9 | V10 | V11 | V12 |
| Assessments | Screening | Baseline^{b} | D1 | D2 | D3 | D4 | D5 | D6 | D7 | D8 | D9 | D10 |
| Informed consent^{d} | X | | | | | | | | | | | |
| Medical history | X | | | | | | | | | | | |
| (demographics) | | | | | | | | | | | | |
| Prior medication | X | | | | | | | | | | | |
| history | | | | | | | | | | | | |
| Inclusion and | X | | | | | | | | | | | |
| exclusion criteria | | | | | | | | | | | | |
| Randomization | | | | | X^{e} | | | | | | | |
| Clinical laboratory | X | X | | | | | | | | | | |
| tests (hematology, | | | | | | | | | | | | |
| biochemistry, | | | | | | | | | | | | |
| urinalysis) | | | | | | | | | | | | |
| Spleen sonography | | X | | | | | | | | | | |
| Urine pregnancy | X | | | | | | | | | | | |
| test (β-hCG)^{f} | | | | | | | | | | | | |
| Physical | X | X^{e} | | | | | | | | | | |
| Examination | | | | | | | | | | | | |
| ECG | | X^{g} | | | | | X | | | | | |
| Vital signs | X | X | X | | | | | | | | | |
| CTX admin.^{h} | | | X | X | X | | | | | | | |
| Bal admin.ⁱ | | | | | | X | | | | | | |
| Filgrastim admin.^{j} | | | | | | X | X | X | X | X | | |
| PD assessments^{k} | | | | | | X | X | | X | | X | |
| PK sampling^{l} | | | | | | X | X | | X | | X | |
| Immunogenicity | | | | | | X^{m} | | | | | | |
| tests | | | | | | | | | | | | |
| Weight | | X | X | | | | | | | | | |
| Adverse event^{a} | | X | X | X | X | X | X | X | X | X | X | X |
| Concomitant | X | X | X^{o} | | | | | | | | | |
| medications | | | | | | | | | | | | |
| Local tolerability^{q} | | | | | X | | | | | | | |
| Temperature^{r} | X | X | X | X | X | X | X | X | X | X | X | X |
| Survival and tumor | | | | | | | | | | | | |
| progression | | | | | | | | | | | | |
| assessment | | | | | | | | | | | | |
| Height | | X | | | | | | | | | | |

| | Open-label treatment period/ | | | | | | | | | |
|---|---|---|---|---|---|---|---|---|---|---|
| | (Cycles 1 through 4) (visit/week) | | | | | | | | Follow-up^{a} | |
| Procedures | V13 | V14 | V15 | V16 | V17 | V23 | V24 | V25 | V26 | V27 |
| Assessments | D11 | D12 | D13 | D14 | D15 | D21 | D24 | EOS^{c} | D180 | D365 |
| Informed consent^{d} | | | | | | | | | | |
| Medical history | | | | | | | | | | |
| (demographics) | | | | | | | | | | |
| Prior medication | | | | | | | | | | |
| history | | | | | | | | | | |
| Inclusion and | | | | | | | | | | |
| exclusion criteria | | | | | | | | | | |
| Randomization | | | | | | | | | | |
| Clinical laboratory | | | | | | | X | | | |
| tests (hematology, | | | | | | | | | | |
| biochemistry, | | | | | | | | | | |
| urinalysis) | | | | | | | | | | |
| Spleen sonography | | | | | | X | | X | | |
| Urine pregnancy | | | | | | | | | | |
| test (β-hCG)^{f} | | | | | | | | | | |
| Physical | | | | | | | X | | | |
| Examination | | | | | | | | | | |
| ECG | | | | | | | | X | | |
| Vital signs | | | | | | | X | | | |
| CTX admin.^{h} | | | | | | | | | | |
| Bal admin.ⁱ | | | | | | | | | | |
| Filgrastim admin.^{j} | | | | | | | | | | |
| PD assessments^{k} | X | | X | | X | | | X | | |
| PK sampling^{l} | | | | | | | | | | |
| X | | X | | | | | | | | |
| Immunogenicity | | | | | | | | | | |
| | | | | | | | X | X | X | |
| tests | | | | | | | | | | |
| Weight | | | | | | | | | | |
| | | | | | | | X | X | X | |
| Adverse event^{a} | X | X | X | X | X | X | X | X | X | X |
| Concomitant | | | | | | | X | | X^{p} | X |
| medications | | | | | | | | | | |
| Local tolerability^{q} | | | | | | | | | | |
| Temperature^{r} | X | X | X | X | X | X | | X | | |
| Survival and tumor | | | | | | | | | X | X |
| progression | | | | | | | | | | |
| assessment | | | | | | | | | | |
| Height | | | | | | | | X | X | X |

^{a}Follow-up period is up to 365 days (12 months, 48 weeks) from the start of the first chemotherapy administration. At the follow-up visits to be performed at Days 180 and 365 after balugrastim first administration, concomitant medication (with regards to G-CSF only) will be documented.
^{b}If the screening visit takes place 5 days prior to the first chemotherapy administration, baseline and screening visits can be combined. Otherwise, they have to be performed separately. Baseline visit assessments can be performed at the D1 visit if all laboratory results will be available before CTX administration.
^{c}End of study visit (EOS) will be performed 4 weeks after the last chemotherapy.
^{e}Abbreviated physical examination only.
^{e}Patients' parents or legal representatives will sign the informed consent.
^{e}Pre-dose of study drug administration (balugrastim or filgrastim).
^{f}Testing will be conducted in female patients with documented menstrual cycle.
^{g}Electrocardiogram (ECG): at baseline (3 triplicates [triplicate recordings will be taken 1 minute apart from each other, there will be an interval of 5 minutes in between each triplicate set]), 24 hours after balugrastim/filgrastim administration at each chemotherapy cycle, and at the end of the study visit.
^{h}VIDE chemotherapy regimen is to be administered on more than one day and differently between cycles. See Table 2 Table for details.
ⁱBalugrastim administration will be approximately 24 h ± 3 h after the end of the last chemotherapy administration for up to 4 cycles.
^{j}Filgrastim will be administered at a dose of 5 μg/kg SC once a day for at least 5 consecutive days or until absolute neutrophil count (ANC) has returned to ≧2 × 10⁹/L for each chemotherapy cycle up to 4 cycles. The maximum period of filgrastim administration is 14 days in each cycle.
^{k}See also Table 4b for a detailed schedule of pharmacodynamic (PD) assessments in relation to study drug administration (balugrastim or filgrastim).
^{l}See also Table 4a for a schedule of pharmacokinetic (PK) assessments in relation to study drug administration (balugrastim).
^{m}Blood samples for immunogenicity assessments will be taken prior to the first administration of balugrastim in Cycle 1 and Cycle 4 (pre-dose). Filgrastim subjects will not undergo immunogenicity testing.
ⁿAE reporting at 6 and 12 months would be limited only to AEs that the Investigator assess as related to study drug.
^{o}Pre-dose to chemotherapy.
^{p}The use of G-CSF will be monitored at Days 180 and 365.
^{q}Tolerability will be assessed 1 h (±10 min) and 24 h (±1 h) following balugrastim or filgrastim first administration at each cycle.
^{r}Temperature will be measured at screening, baseline, Days 1 to 15, and 21 of chemotherapy cycle, and at end of study visit (EOS).
admin. = administration;
Balu = balugrastim;
β-hCG = β human chorionic gonadotropin;
CTX = chemotherapy;
D = day;
ECG = electrocardiography;
V = visit.

TABLE 6
Schedule of Assessments - VDC/IE Chemotherapy Regimen

| | Pretreatment | | Open-label treatment period/(Cycles 1 through 4) (visit/week) | | | | | | | | | | |
|---|---|---|---|---|---|---|---|---|---|---|---|---|---|
| Procedures | V1 | V2 | V3 | V4 | V5 | V6 | V7 | V8 | V9 | V10 | V11 | V12 | V13 |
| Assessments | Screening | Baseline^{b} | D1 | D2 | D3 | D4 | D5 | D6 | D7 | D8 | D9 | D10 | D11 |
| Informed | X | | | | | | | | | | | | |
| consent^{d} | | | | | | | | | | | | | |
| Medical history | X | | | | | | | | | | | | |
| (demographics) | | | | | | | | | | | | | |
| Prior medication | X | | | | | | | | | | | | |
| history | | | | | | | | | | | | | |
| Inclusion and | X | | | | | | | | | | | | |
| exclusion criteria | | | | | | | | | | | | | |
| Randomization | | | | X^{c} | | | | | | | | | |
| Clinical | X | X | | | | | | | | | | | |
| laboratory tests | | | | | | | | | | | | | |
| (hematology, | | | | | | | | | | | | | |
| biochemistry, | | | | | | | | | | | | | |
| urinalysis) | | | | | | | | | | | | | |
| Spleen | | X | | | | | | | | | | | |
| sonography | | | | | | | | | | | | | |
| Urine pregnancy | X | | | | | | | | | | | | |
| test (β-hCG)^{f} | | | | | | | | | | | | | |
| Physical | X | X^{g} | | | | | | | | | | | |
| examination | | | | | | | | | | | | | |
| ECG | | X^{h} | | | | X | | | X | | | | |
| Vital signs | X | X | X | | | | | | | | | | |
| CTX admin.ⁱ | | | X | X | X | X | X | | | X | | | |
| Bal admin. on | | | | | X | | | | | | | | |
| cycles 1 and 3^{j} | | | | | | | | | | | | | |
| Bal admin. on | | | | | | | | X | | | | | |
| cycles 2 and 4 | | | | | | | | | | | | | |
| Filgrastim | | | | | X | X | X | X | X | | | | |
| admin. on | | | | | | | | | | | | | |
| cycles 1 and 3^{k} | | | | | | | | | | | | | |
| Filgrastim | | | | | | | | X | X | X | X | X | |
| admin. on | | | | | | | | | | | | | |
| cycles 2 and 4 | | | | | | | | | | | | | |
| PD assessments | | | | | X | X | | X | | X | | X | |
| on cycles 1 and 3^{l} | | | | | | | | | | | | | |
| PD assessments | | | | | | | | X | X | | X | | X |
| on cycles 2 and 4 | | | | | | | | | | | | | |
| PK sampling | | | | | X | X | | X | | X | | X | |
| cycles 1 and 3^{m} | | | | | | | | | | | | | |
| PK sampling | | | | | | | | X | X | | X | | X |
| cycles 2 and 4^{m} | | | | | | | | | | | | | |
| Immunogenicity | | | | | Xⁿ | | | X | | | | | |
| tests | | | | | | | | | | | | | |
| Weight | | X | X | | | | | | | | | | |
| Adverse events^{o} | | X | X | X | X | X | X | X | X | X | X | X | X |
| Concomitant | X | X | X^{p} | | | | | | | | | | |
| medications | | | | | | | | | | | | | |
| Local tolerability | | | | | X^{r} | | | X^{s} | | | | | |
| Temperature^{t} | X | X | X | X | X | X | X | X | X | X | X | X | X |
| Survival and | | | | | | | | | | | | | |
| tumor | | | | | | | | | | | | | |
| progression | | | | | | | | | | | | | |
| assessment | | | | | | | | | | | | | |
| Height | | X | | | | | | | | | | | |

| | Open-label treatment period/(Cycles 1 through 4) (visit/week) | | | | | | | | | Follow-up^{a} | |
|---|---|---|---|---|---|---|---|---|---|---|---|
| Procedures | V14 | V15 | V16 | V17 | V18 | V19 | V23 | V24 | V25 | V26 | V27 |
| Assessments | D12 | D13 | D14 | D15 | D16 | D17 | D21 | D24 | EOS^{c} | D180 | D365 |
| Informed | | | | | | | | | | | |
| consent^{d} | | | | | | | | | | | |
| Medical history | | | | | | | | | | | |
| (demographics) | | | | | | | | | | | |
| Prior medication | | | | | | | | | | | |
| history | | | | | | | | | | | |
| Inclusion and | | | | | | | | | | | |
| exclusion criteria | | | | | | | | | | | |
| Randomization | | | | | | | | | | | |
| Clinical | | | | | | | | X | | | |
| laboratory tests | | | | | | | | | | | |
| (hematology, | | | | | | | | | | | |
| biochemistry, | | | | | | | | | | | |
| urinalysis) | | | | | | | | | | | |
| Spleen | | | | | | | X | | X | | |
| sonography | | | | | | | | | | | |
| Urine pregnancy | | | | | | | | | | | |
| test (β-hCG)^{f} | | | | | | | | | | | |
| Physical | | | | | | | | X | | | |
| examination | | | | | | | | | | | |
| ECG | | | | | | | | | X | | |
| Vital signs | | | | | | | | X | | | |
| CTX admin.ⁱ | | | | X | | | | | | | |
| Bal admin. on | | | | | | | | | | | |
| cycles 1 and 3^{j} | | | | | | | | | | | |
| Bal admin. on | | | | | | | | | | | |
| cycles 2 and 4 | | | | | | | | | | | |
| Filgrastim | | | | | | | | | | | |
| admin. on | | | | | | | | | | | |
| cycles 1 and 3^{k} | | | | | | | | | | | |
| Filgrastim | | | | | | | | | | | |
| admin. on | | | | | | | | | | | |
| cycles 2 and 4 | | | | | | | | | | | |
| PD assessments | X | | X | | | | | | X | | |
| on cycles 1 and 3 | | | | | | | | | | | |
| PD assessments | | X | | X | | X | | | X | | |
| on cycles 2 and 4 | | | | | | | | | | | |
| PK sampling | X | | | | | | | | | | |
| cycles 1 and 3^{m} | | | | | | | | | | | |
| PK sampling | | X | | X | | | | | | | |
| cycles 2 and 4^{m} | | | | | | | | | | | |
| Immunogenicity | | | | | | | | | X | X | X |
| tests | | | | | | | | | | | |
| Weight | | | | | | | | | X | X | X |
| Adverse events^{o} | X | X | X | X | X | X | X | X | X | X | X |
| Concomitant | | | | | | | | X | | X^{q} | X |
| medications | | | | | | | | | | | |
| Local tolerability | | | | | | | | | | | |
| Temperature^{t} | X | X | X | X | | | X | | X | | |
| Survival and | | | | | | | | | | X | X |
| tumor | | | | | | | | | | | |
| progression | | | | | | | | | | | |
| assessment | | | | | | | | | | | |
| Height | | | | | | | | | X | X | X |

^{a}Follow-up period is up to 365 days (12 months, 48 weeks) from the start of the first chemotherapy administration. At the follow-up visits to be performed at Days 180 and 365 after balugrastim first administration, concomitant medication (with regards to G-CSF only) will be documented.
^{b}If the screening visit takes place 5 days prior to the first chemotherapy administration, baseline and screening visits can be combined. Otherwise, they have to be performed separately. Baseline visit assessments can be performed at the D1 visit if all laboratory results will be available before CTX administration.
^{c}End of study visit (EOS) will be performed 4 weeks after the end of the last chemotherapy.
^{d}Patients' parents or legal representatives will sign the informed consent.
^{e}Pre-dose of study drug administration (balugrastim or filgrastim).
^{f}Testing will be conducted in female patients with documented menstrual cycle.
^{g}Abbreviated physical examination only.
^{h}Electrocardiogram (ECG): at baseline (3 triplicates [triplicate recordings will be taken 1 minute apart from each other, there will be an interval of 5 minutes in between each triplicate set]), 24 hours after balugrastim or filgrastim administration at each chemotherapy cycle. ECG will be taken on Day 4 of Cycles 1 and 3, and on Day 7 of Cycles 2 and 4, and at the end of the study visit.
ⁱVDC/IE chemotherapy regimen is to be administered on more than one day and differently between cycles. See Table 2 for details.
^{j}Balugrastim administration will be approximately 24 h ± 3 h after the end of the last chemotherapy administration for up to 4 cycles.
^{k}Filgrastim will be administered at a dose of 5 μg/kg SC once a day for at least 5 consecutive days or until absolute neutrophil count (ANC) has returned to ≧2 × 10⁹/L for each chemotherapy cycle up to 4 cycles. The maximum period of filgrastim administration is 14 days in each cycle.
^{l}See also Table 4b for a detailed schedule of pharmacodynamic (PD) assessments in relation to study drug administration (balugrastim or filgrastim).
^{3m}See also Table 4a for a schedule of pharmacokinetic (PK) assessments in relation to study drug administration (balugrastim).
ⁿBlood samples for immunogenicity assessments will be taken prior to the first administration of balugrastim in Cycle 1 and Cycle 4 (pre-dose). Filgrastim subjects will not undergo immunogenicity testing.
^{o}AE reporting at 6 and 12 months would be limited only to AEs that the Investigator assess as related to study drug.
^{p}Pre-dose to chemotherapy.
^{q}The use of G-CSF will be monitored at Days 180 and 365.
^{r}Tolerability will be assessed 1 h (±10 min) and 24 h (±1 h) following balugrastim or filgrastim first administration on Day 3 during Cycles 1 and 3.
^{s}Tolerability will be assessed 1 h (±10 min) and 24 h (±1 h) following balugrastim or filgrastim first administration on Day 6 during Cycles 2 and 4.
^{t}Temperature will be measured at screening, baseline, Days 1 TO 15, and 21 of chemotherapy cycle and at end of study visit (EOS).
admin. = administration;
Balu = balugrastim;
β-hCG = β human chorionic gonadotropin;
CTX = chemotherapy;
D = day;
ECG = electrocardiography;
V = visit

TABLE 7
Pharmacodynamics and Pharmacokinetics Assessments

| | PKa assessments (Cycle 1) | | | | | | | | | | | | |
|---|---|---|---|---|---|---|---|---|---|---|---|---|---|
| | (Hours post-injection) | | | | | | | | PD Assessmentsb (Cycles 1 to 4) | | | | |
| | −1 | | | | | | | | −1 | | Day 2 | D4-D12 | |
| | (pre- | | | 24 | 72 | 120 | 168 | 216 | (pre- | | following | following | |
| Procedure | dose) | 6 | 12 | (1 day) | (3 days) | (5 days) | (7 days) | (9 days) | dose) | D1 | SD admin. | SD admin. | EOS |
| PK | X | X | X | X | X | X | X | X | | | | | |
| PD^{c} | | | | | | | | | X | X | X | Every other | X |
| | | | | | | | | | | | | day | |

^{a}Balugrastim will be administered SC approximately 24 hours (±3) after the end of the last chemotherapy administration in Week 1 of the specific regimen (VDC/IE regimen: chemotherapy Day 3 during Cycles 1 and 3, and on chemotherapy Day 6 during Cycles 2 and 4; VIDE regimen: chemotherapy Day 4; IVA and IVAd regimen: Day 3), for up to 4 cycles.
^{b}Balugrastim will be administered SC approximately 24 hours (±3) after the end of the last chemotherapy administration in Week 1 of the specific regimen (VDC/IE regimen: chemotherapy Day 3 during Cycles 1 and 3, and on chemotherapy Day 6 during Cycles 2 and 4; VIDE regimen: chemotherapy Day 4 IVA and IVAd regimen: Day 3), for up to 4 cycles.
^{c}Samples for PD assessments will be obtained 1 hour prior to study drug administration during Cycles 1-4 on Day 1 (VDC/IE regimen: Day 3 during Cycles 1 and 3, and Day 6 during Cycles 2 and 4; VIDE regimen: Day 4; IVA and IVAd regimen: Day 3), and then at least every other day starting on Day 2 to Day 12 following drug administration (VDC/IE regimen: Days 4 to 14 during Cycles 1 and 3, and Days 7 to 17 during Cycles 2 and 4; VIDE regimen: Days 5 to 15; IVA and IVAd regimen: Days 4 to 14) until ANC ≧2 × 10⁹/L after nadir, and then twice weekly during the respective cycle or the EOS
admin = administration;
D = day;
SD = study drug.

Prior to performing any study activities/evaluations, the patient and the patient's parent(s) or legal representative(s) must be thoroughly informed about all aspects of the study, including scheduled study visits and activities. The patient's parent(s) or legal representative(s) must sign the informed consent. In addition, assent is obtained from the patient, depending on the development stage, intellectual capacities, life/disease experience, etc. and in accordance with local regulatory requirements. A signed copy of the informed consent (and assent form if applicable) should be given to the patient and the patient's parent (s) or legal representative(s).

Every effort should be made to understand and respect differences of opinion between the child and his/her parent(s) or legal representative(s) regarding study participation. Strong and definitive objections from the child should be respected.

All screening procedures must be completed within 14 days prior to the first day of the first chemotherapy cycle (Day 1).

The following procedures is performed during screening, irrespective of the chemotherapy regimen (VIDE, VDC/IE, IVA, or IVAd):
informed consent
medical history (demographics)
prior medication history
evaluation of eligibility (inclusion and exclusion criteria)
clinical laboratory tests (hematology, biochemistry and urinalysis)
urine pregnancy test for female patients who have attained menarche
physical examination
vital signs
concomitant medications
temperature

If chemotherapy is not started 14 days after screening due to medical or administrative reasons, the patient can be re-screened. In this case, screening procedures should be repeated.

Procedures Before Starting Chemotherapy (Baseline [Visit 2])

Irrespective of the chemotherapy regimen (VIDE, VDC/IE, IVA, or IVAd), patients are scheduled for visits to the clinic before the study treatment period for the following:
clinical laboratory tests (hematology, biochemistry and urinalysis)
spleen sonography
physical examination
12 lead ECG (3 triplicates)
vital signs
weight
AE evaluation
concomitant medications
temperature
height

Procedures During Study Drug Treatment Throughout the treatment period, close monitoring for liver-related AEs (e.g., jaundice) is performed. If such symptoms occur, blood testing for liver enzymes is performed.

VIDE Chemotherapy Regimen

The scheduled assessments for the VIDE chemotherapy regimen are presented in Table 5. Chemotherapy begins on Day 1 of Cycle 1. This regimen is administered on more than 1 day and differently between cycles, see Table 2 for details. Study drug (balugrastim or filgrastim) is administered at approximately 24 hours (±3 hours) after the end of the last chemotherapy in Week 1.

During the treatment period, blood samples for balugrastim PK assessments are taken only in Cycle 1. Samples are taken in relation to the day of balugrastim administration according to the different chemotherapy regimens as indicated in Table 3. The pre-dose samples are taken within 1 hour prior to the first injection of balugrastim on Day 1 followed by sampling at 6, 12, 24 hours (1 day) post-injection. Subsequent samples are taken 72 (3 days), 120 (5 days), 168 (7 days), and 216 (9 days) hours post-injection of balugrastim in Cycle 1.

Blood samples for PD assessments are taken prior to study drug administration in Cycles 1 to 4, on Day 1 of study drug administration, and at least every other day on Days 2 to 12 following study drug administration until ANC≧2.0×10⁹/L, and then twice weekly during the respective cycle or the EOS (see Table 7). For the VIDE regimen, PD samples are taken on chemotherapy Day 4, and on Days 5, 7, 9, 11, 13, 15, and EOS.

Samples are obtained from an indwelling catheter to minimize discomfort to the patients.

AE evaluations are ongoing. Other safety assessments are scheduled periodically throughout the study.

Assessment and procedures to be conducted on any specific day are listed in this section.

Chemotherapy Cycle 1, Day 1, Week 1

The following procedures are performed on the first day of the patient's chemotherapeutic regimen:
vital signs
chemotherapy administration
weight
AEs evaluation
concomitant medications
temperature

Chemotherapy Cycle 1, Day 2, Week 1
chemotherapy administration
AEs evaluation
temperature

Chemotherapy Cycle 1, Last Day of Chemotherapy in Week 1 (Day 3)
randomization
chemotherapy administration
AEs evaluation
temperature

Chemotherapy Cycle 1, Day 4 (next day after Chemotherapy), Week 1
balugrastim or filgrastim administration (24±3 h) after the last chemotherapy administration in Week 1. The following procedures are performed on the day of balugrastim or filgrastim administration:
   PD assessment (for ANC) obtained prior (within 1 hour) to balugrastim or filgrastim administration
   PK assessments
   immunogenicity tests (samples are obtained prior to balugrastim administration)
   AEs evaluation
   local tolerability
   temperature

Chemotherapy Cycle 1, Day 5, Week 1

One day (alternatively, two days) after study drug administration:
ECG
filgrastim administration (only to the group receiving filgrastim)
local tolerability
PD assessments
PK assessments
AEs evaluation
temperature

Chemotherapy Cycle 1, Day 6, Week 1
filgrastim administration (only to the group receiving filgrastim)
AEs evaluation
temperature

Chemotherapy Cycle 1, Day 7, Week 1
filgrastim administration (only to the group receiving filgrastim)
PD assessments
PK assessments
AEs evaluation
temperature

Chemotherapy Cycle 1, Day 8, Week 2
filgrastim administration (only to the group receiving filgrastim)
AEs evaluation
temperature

Chemotherapy Cycle 1, Day 9, Week 2
PD assessments
PK assessments
AEs evaluation
temperature

Chemotherapy Cycle 1, Day 10, Week 2
AEs evaluation
temperature

Chemotherapy Cycle 1, Day 11, Week 2
PD assessments
PK assessments
AEs evaluation
temperature

Chemotherapy Cycle 1, Day 12, Week 2
AEs evaluation
temperature

Chemotherapy Cycle 1, Day 13, Week 2
PD assessments
PK assessments
AEs evaluation
temperature

Chemotherapy Cycle 1, Day 14, Week 2
AEs evaluation
temperature

Chemotherapy Cycle 1, Day 15, Week 3
PD assessments
AEs evaluation
temperature

Chemotherapy Cycle 1, Day 17 or Day 21
spleen sonography
AEs evaluation
temperature

Chemotherapy Cycle, Day 17 or End of treatment, Day 24
clinical laboratory tests (hematology, biochemistry, urinalysis)
physical examination
vital signs
AEs evaluation
concomitant medication

Before proceeding to the next cycle of chemotherapy the following should be met:
WBC count: >2.5×10⁹/L
ANC: ≧1.5×10⁹/L
Platelet count≧100×10⁹/L

EOS Visit (4 weeks after the start of the last chemotherapy cycle±3 days)
spleen sonography
12 lead ECG (1 triplicate)
PD assessments
immunogenicity tests
weight
AEs evaluation
temperature
height

VDC/IE Chemotherapy Regimen

The scheduled assessments for the VDC/IE chemotherapy regimen are presented in Table 6. Chemotherapy begins on Day 1 of Cycle 1. This regimen is administered on more than 1 day and differently between cycles, see Table 2 for details. Study drug (balugrastim or filgrastim) is administered approximately 24 h±3 h after the end of the last chemotherapy administration for up to 4 cycles. Balugrastim is administered on Day 3 during Cycles 1 and 3, and on Day 6 during Cycles 2 and 4. Filgrastim is administered on Days 3 to 7 during Cycles 1 and 3, and on Days 6 to 10 during Cycles 2 and 4.

During the treatment period, blood samples for balugrastim PK assessments are taken only in Cycle 1. Samples are taken in relation to the day of balugrastim administration as indicated in Table 3. The pre dose sample are taken within 1 hour prior to the first injection of balugrastim on Day 1 followed by sampling at 6, 12 and 24 hours post injection. Subsequent samples are taken on Days 3, 5, 7 and 9 in Cycle 1 after balugrastim administration.

Blood samples for PD assessments are taken prior to study drug administration in Cycles 1 to 4, on Day 1 of study drug administration, and at least every other day on Days 2 to 12 following study drug administration until ANC≧2.0×10⁹/L, and then twice weekly during the respective cycle or the EOS (see Table 7). For the VDC/IE regimen, PD samples are taken on chemotherapy Day 3 and every other day on chemotherapy Days 4 to 14 during Cycles 1 and 3; and on chemotherapy Day 6 and every other day on chemotherapy Days 7 to 17 during Cycles 2 and 4.

Samples are obtained from an indwelling catheter to minimize discomfort to the patients.

AE evaluations are ongoing. Other safety assessments are scheduled periodically throughout the study.

Assessments and procedures to be conducted on any specific day are listed in this section.

Chemotherapy Cycle 1, Day 1, Week 1

The following procedures are performed on the first day of the patient's chemotherapeutic regimen:
vital signs
chemotherapy administration
weight
AEs evaluation
concomitant medications
temperature

Chemotherapy Cycle 1, Day 2, Week 1
randomization
chemotherapy administration
AEs evaluation
temperature

Chemotherapy Cycle 1, Day 3, Week 1
chemotherapy administration (e.g., on Cycles 2 and 4)
balugrastim or filgrastim administration on Cycles 1 and 3, study drugs are administered 24±3 h after the last chemotherapy administration
PD assessments on Cycles 1 and 3
PK assessments on Cycle 1 (alternatively, Cycles 1 and 3)
AEs evaluation
temperature
local tolerability on Cycles 1 and 3
immunogenicity on Cycle 1 (alternatively on Cycles 1 and 3) (samples will be obtained within 8 hours prior to balugrastim administration)

Chemotherapy Cycle 1, Day 4, Week 1
ECG (1 triplicate) after study drug administration (e.g., on Cycles 1 and 3)
chemotherapy administration (e.g., on Cycles 2 and 4)
filgrastim administration on Cycles 1 and 3
local tolerability on Cycles 1 and 3
PD assessment on Cycles 1 and 3
PK assessments on Cycles 1 and 3
AEs evaluation
temperature

Chemotherapy Cycle 1, Day 5, Week 1
chemotherapy administration (e.g., on Cycles 2 and 4)
filgrastim administration on Cycles 1 and 3
AEs evaluation
temperature

Chemotherapy Cycle 1, Day 6, Week 1
balugrastim administration on Cycles 2 and 4
filgrastim administration on Cycles 1 and 3
filgrastim administration on Cycles 2 and 4
PD assessments on Cycles 1 and 3
PD assessments on Cycles 2 and 4
PK assessments on Cycle 1 (alternatively on Cycles 1 and 3)
Optionally, PK assessments on Cycles 2 and 4
AEs evaluation
local tolerability on Cycles 2 and 4
temperature
immunogenicity on Cycle 4 (alternatively on Cycles 2 and 4) (samples will be obtained within 8 hours prior to balugrastim administration)

Chemotherapy Cycle 1, Day 7, Week 1
ECG (e.g, on Cycles 2 and 4)
filgrastim administration on Cycles 1 and 3
filgrastim administration on Cycles 2 and 4
PD assessments on Cycles 2 and 4
Optionally, PK assessments on Cycles 2 and 4
AEs evaluation
local toleratibility on Cycles 2 and 4
temperature

Chemotherapy Cycle 1, Day 8, Week 2
chemotherapy administration (e.g., on Cycles 1 and 3)
filgrastim administration on Cycles 2 and 4
PD assessments on Cycles 1 and 3
PK assessments on Cycle 1 (alternatively on Cycles 1 and 3)
AEs evaluation
temperature

Chemotherapy Cycle 1, Day 9, Week 2
filgrastim administration on Cycles 2 and 4
PD assessments on Cycles 2 and 4
Optionally, PK assessments on Cycles 2 and 4
AEs evaluation
temperature

Chemotherapy Cycle 1, Day 10, Week 2
filgrastim administration on Cycles 2 and 4
PD assessments on Cycles 1 and 3
PK assessments on Cycle 1 (alternatively on Cycles 1 and 3)
AEs evaluation
temperature

Chemotherapy Cycle 1, Day 11, Week 2
PD assessments on Cycles 2 and 4
Optionally, PK assessments on Cycles 2 and 4
AEs evaluation
temperature

Chemotherapy Cycle 1, Day 12, Week 2
PD assessments on Cycles 1 and 3
PK assessments on Cycle 1 (alternatively, on Cycles 1 and 3)
AEs evaluation
temperature

Chemotherapy Cycle 1, Day 13, Week 2
PD assessments on Cycles 2 and 4
Optionally, PK assessments on Cycles 2 and 4
AEs evaluation
temperature

Chemotherapy Cycle 1, Day 14, Week 2
PD assessments on Cycles 1 and 3
AEs evaluation
temperature

Chemotherapy Cycle 1, Day 15, Week 3
chemotherapy administration (e.g., on Cycles 1 and 3)
PD assessments on Cycles 2 and 4
Optionally, PK assessments on Cycles 2 and 4
AEs evaluation
temperature

Chemotherapy Cycle 1, Day 16, Week 3
AEs evaluation

Chemotherapy Cycle 1, Day 17, Week 3
PD assessments on Cycles 2 and 4
AEs evaluation

Chemotherapy Cycle 1, Day 17 (or Day 21)
spleen sonography
AEs evaluation
temperature

End of treatment, Day 17 (or Day 24)
clinical laboratory tests (hematology, biochemistry, urinalysis)
physical examination
vital signs
AEs evaluation
concomitant medication

Before proceeding to the next cycle of chemotherapy the following should be met:
WBC count: >2.5×10⁹/L
ANC: ≧1.5×10⁹/L
Platelet count≧100×10⁹/L

EOS Visit (4 weeks after the end of the last cycle)
spleen sonography
12 lead ECG (1 triplicate)
PD assessments
immunogenicity tests
weight
AEs evaluation
temperature
height

### Follow Up Period Procedures

Follow-up visits for both chemotherapeutic regimens, VIDE and VDC/IE, take place approximately at Day 180 and Day 365 from the start of the first chemotherapy administration. The following is assessed during the follow up visits:
immunogenicity tests
weight
AEs evaluations (VIDE)
concomitant medications (only G CSF)
survival and tumor/metastases progression assessment
height

IVA and IVAd Chemotherapy Regimens

Chemotherapy begins on Day 1 of Cycle 1. This regimen is administered on more than 1 day and differently between cycles, see Table 2 for details. Study drug (balugrastim or filgrastim) is administered approximately 24 h±3 h after the end of the last chemotherapy administration for up to 4 cycles. Balugrastim is administered on Day 3 during each cycle. Filgrastim is administered on Days 3 to 7 during each cycle.

During the treatment period, blood samples for balugrastim PK assessments are taken only in Cycle 1. Samples are taken in relation to the day of balugrastim administration as indicated in Table 3. The pre-dose sample is taken within 1 hour prior to the first injection of balugrastim on Day 3 followed by sampling at 6, 12, and 24 hours (Day 4) post-injection. Subsequent samples are taken on Days 36, 8, 10, and 12 in Cycle 1. In total, 8 samples are taken in Cycle 1.

Blood samples for PD assessments are taken prior to study drug administration in Cycles 1 to 4, on Day 1 of study drug administration, and at least every other day on Days 2 to 12 following study drug administration until ANC≧2.0×10⁹/L, and then twice weekly during the respective cycle or the EOS (see Table 7). For the IVA and IVAd regimens, PD samples are taken on chemotherapy Day 3 and every other day on chemotherapy Days 4 to 14. In total, 29 samples are taken.

Samples are obtained from an indwelling catheter to minimize discomfort to the patients.

AE evaluations are ongoing. Other safety assessments are scheduled periodically throughout the study.

Assessments and procedures to be conducted on any specific day are listed in this section.

Chemotherapy Cycle, Day 1, Week 1

The following procedures are performed on the first day of the patient's chemotherapeutic regimen:
vital signs
chemotherapy administration
weight
AEs evaluation
concomitant medications
temperature

Chemotherapy Cycle, Day 2, Week 1
randomization
chemotherapy administration
AEs evaluation
temperature

Chemotherapy Cycle, Day 3, Week 1
balugrastim or filgrastim administration; study drugs are administered 24±3 h after the last chemotherapy administration
PD assessments
PK assessments on Cycle 1
AEs evaluation
temperature
local tolerability on Cycles 1 and 3
immunogenicity (samples will be obtained within 8 hours prior to balugrastim administration)

Chemotherapy Cycle, Day 4, Week 1
ECG (1 triplicate) after study drug administration
filgrastim administration
local tolerability
PD assessment
PK assessments on Cycle 1
AEs evaluation
temperature

Chemotherapy Cycle, Day 5, Week 1
filgrastim administration
AEs evaluation
temperature

Chemotherapy Cycle, Day 6, Week 1
filgrastim administration
PD assessments
PK assessments on Cycle 1
AEs evaluation
temperature

Chemotherapy Cycle, Day 7, Week 1
filgrastim administration
AEs evaluation
temperature

Chemotherapy Cycle, Day 8, Week 2
chemotherapy administration (except Cycle 3)
PD assessments
PK assessments
AEs evaluation
temperature

Chemotherapy Cycle, Day 9, Week 2
AEs evaluation
temperature

Chemotherapy Cycle, Day 10, Week 2
PD assessments
PK assessments on Cycle 1
AEs evaluation
temperature

Chemotherapy Cycle, Day 11, Week 2
AEs evaluation
temperature

Chemotherapy Cycle, Day 12, Week 2
PD assessments
PK assessments on Cycle 1
AEs evaluation
temperature

Chemotherapy Cycle, Day 13, Week 2
AEs evaluation
temperature

Chemotherapy Cycle, Day 14, Week 2
PD assessments
AEs evaluation
temperature

Chemotherapy Cycle, Day 15, Week 3
chemotherapy administration (except cycle 3)
AEs evaluation
temperature

Chemotherapy Cycle, Day 16, Week 3
AEs evaluation

Chemotherapy Cycle, Day 17
spleen sonography
AEs evaluation
temperature
clinical laboratory tests (hematology, biochemistry, urinalysis)
physical examination
vital signs
concomitant medication

Before proceeding to the next cycle of chemotherapy the following should be met:
WBC count: >2.5×10⁹/L
ANC: ≧1.5×10⁹/L
Platelet count≧100×10⁹/L

EOS Visit (4 weeks after start of the last chemotherapy cycle±3 days)
spleen sonography
12-lead ECG (1 triplicate)
PD assessments
immunogenicity tests
weight
AEs evaluation
temperature
height

### Follow Up Period Procedures

Follow-up visits for all chemotherapeutic regimens, VIDE, VDC/IE, IVA, and IVAd take place approximately at Day 180 and Day 365 from the start of the first chemotherapy administration. The following is assessed during the follow up visits:
immunogenicity tests
weight
Optionally, AEs evaluations (VIDE)
concomitant medications (only G-CSF)
survival and tumor/metastases progression assessment
height

Procedures After Study Drug Treatment

The reason for discontinuation of the study is documented in the source documents and captured on the CRF. In the event that the patient discontinues the study prior to completion of the PK and PD assessments, an EOS visit should be completed as close to the last study assessment as possible.

EOS assessments include: spleen sonography, 12 lead ECG, immunogenicity test, weight, AE assessment, temperature, and height.

If a patient is withdrawn because of an AE, the appropriate “Withdrawal Section” of the CRFs should be fully completed in addition to the AE module. The Sponsor should be informed of all patients who are withdrawn due to an AE.

Documented attempts will be made to follow up a patient who is prematurely discontinued from the study. Patients who have not withdrawn consent should be offered EOS assessments and also invited to have immunogenicity tests at 180 days (±2 weeks) and 365 days (±2 weeks) following last balugrastim administration.

Selection and Withdrawal of Patients

Female or male children and adolescents with solid tumors receiving chemotherapy may be screened for enrolment in the NEUGR 05 study. The study will be conducted in up to 20 sites in up to 8 countries in Bulgaria, Hungary, Russia, Ukraine, Romania, Georgia, Czech Republic and Slovakia.

### Patient Inclusion Criteria

Patients must meet all inclusion criteria in order to be eligible for the study:

a. Histological or cytologically-confirmed solid tumor in a subject for whom the study chemotherapy regimen (VIDE, VDC/IE, IVA, or IVAd) is considered an appropriate treatment.
b. Minimum body weight of 10 kg (alternatively 15 kg)
c. Life expectancy of at least 3 months with appropriate therapy
d. Female or male children and adolescents aged 2 to 17 years
e. Written informed consent provided by parent(s)/legal representative(s) of the pediatric patient and patient's assent if appropriate at the time of screening.
f. Fertile patients (male or female) must use highly reliable contraceptive measures (i.e. two of the following: oral contraception, implants, injections, barrier contraception, and intrauterine device, or vasectomized/sterilized partners, or sexual abstinence). For the purposes of this study, a fertile female patient is any female patient who has experienced menarche and who has not undergone tubal ligation.
g. Female patients who have attained menarche must have a negative urine pregnancy test at the screening visit.

### Patient Exclusion Criteria

Patients are excluded from participating in this study if one or more of the following criteria are met:

a. Primary bone marrow disease
b. Prior radiation therapy within 4 weeks of randomization into the study (alternatively, previous chemotherapy or wide-field irradiation to the pelvis)
c. Previous exposure to filgrastim, pegfilgrastim, lenograstim or other granulocyte-colony stimulating factor (G-CSF) less than 6 months before randomization.
d. Known hypersensitivity to filgrastim, pegfilgrastim or lenograstim
e. Pregnancy or breastfeeding (if a patient becomes pregnant during the study she will be withdrawn from the study).
f. Major surgery, serious infection, serious trauma or compound medical procedure within 3 weeks before the first administration of study drug (or, optionally, within the 4 weeks prior to the first study drug dose).
g. Subjects with a clinically significant or unstable medical or surgical condition that would preclude safe and complete study participation, as determined by medical history, physical exams, ECG, laboratory tests or imaging.

### Treatment of Patients

### Study Drugs Administered

### Study Drug: Balugrastim

300 μg/kg SC once per chemotherapy cycle, approximately 24 h after chemotherapy, up to 4 cycles
670 μg/kg (maximum 40 mg) SC once per chemotherapy cycle, approximately 24 h after chemotherapy, up to 4 cycles

### Comparator: Filgrastim

Filgrastim 5 μg/kg SC once daily for a minimum of 5 consecutive days and a maximum of 14 days or until the ANC has returned to ≧2.0×10⁹/L up to 4 cycles.

### Restrictions

Patients are required to comply with the following restrictions:

### 1. Activity

Patients must remain seated for safety reasons during each study drug administration and for 1 hour following each study drug administration. Patients will be allowed to use the restroom (escorted by study center staff) during this time, as needed.

Patients are not to engage in strenuous exercise during the inpatient periods of the study.

2. Specific food, beverages, etc.

The consumption of alcohol, grapefruit juice, and caffeine (and optionally, the use of antiseptic mouthwash) will not be permitted 48 hours before each dose of study drug and throughout each PK sampling period.

In addition, excessive consumption of coffee, tea, and/or caffeine-containing beverage or food (ie 600 mg of caffeine or more per day, or 5 or more cups of coffee per day) will be prohibited for a minimum of 2 weeks prior to the dose of study drug and throughout the study, including follow-up.

### 3. Medications

Patients may not receive other filgrastim products or lithium during their participation in this study.

### Prior and Concomitant Therapy or Medication

Any prior or concomitant therapy or medication given to a patient 30 days before study drug administration or during study drug administration is indicated on the CRF. Dosage and generic name or trade name are indicated. The Sponsor encodes all therapy and medication according to the World Health Organization (WHO) drug dictionary (WHO Drug).

The following rescue treatments are allowed in this trial:
Combined antibiotics in case of fever according to the standards of the study center
Optionally, Filgrastim for patients randomized to balugrastim

Although they are not medications per se, the following measures are going to be used to minimize pain and distress.

Topical anesthesia is to be offered to all the patients when placing IV catheters.
Already existing indwelling catheters are used for blood sampling rather than repeated vein punctures.
Collection of protocol-specified blood samples are performed when obtaining routine clinical samples.
Microtainers are used with young children to minimize the blood volume taken.

### Assessment of Efficacy

### Primary Efficacy Variable

The primary efficacy variable of this study is the DSN (severe neutropenia defined as ANC<0.5×10⁹/L) in Cycle 1.

### Secondary Efficacy Variables

The secondary efficacy variables in this study are:

### 1. DSN in Cycles 2 to 4;

2. incidence of severe neutropenia in Cycles 1 to 4; and
3. frequency of febrile neutropenia (defined as body temperature>38.5° C. for more than 1 hour [axillary measurements]) and ANC (<0.5×10⁹/L) by cycle and across all cycles.

### Assessment of Safety

In this study, safety is assessed by evaluating reported AEs, clinical laboratory results, vital signs measurements, physical examination findings (including body weight and height measurements), ECG assessment and immunogenicity. Blood samples for immunogenicity testing are taken prior to the administration of chemotherapy in Cycle 1, at the end of study visit and 6 and 12 months after the first injection of balugrastim (Days 180 and 365). In addition, the effect of treatment on mortality due to infections and overall mortality are examined.

### Adverse Events

### Definition of an Adverse Event

An AE is any untoward medical occurrence in a patient that develops or worsens in severity during the conduct of a clinical study of a pharmaceutical product or a device and does not necessarily have a causal relationship to the study drug.

An AE can, therefore, be any unfavorable and unintended physical sign, symptom, or laboratory parameter that develops or worsens in severity during the course of the study, or significant worsening of the disease under study (or any concurrent disease), whether or not considered related to the study drug. A new condition or the worsening of a pre-existing condition will be considered an AE. Stable chronic conditions (such as arthritis) that are present prior to study entry and do not worsen during the study is not considered AEs.

Worsening of the disease under study is expressed by mortality and/or tumor progression and should be recorded as an AE only if the presentation and/or outcome is more severe than would normally be expected from the normal course of the disease in a particular patient, or if a fatal outcome resulted from cancer progression as well as another AE.

Accordingly, an AE could include any of the following:
intercurrent illnesses
physical injuries
events possibly related to concomitant medication
significant worsening (change in nature, severity, or frequency) of the disease under study or other pre-existing conditions. (NOTE: A condition, recorded as pre-existing, that is intermittently symptomatic [eg headache] and which occurs during the study should be recorded as an AE)
drug interactions
events occurring during diagnostic procedures or any washout phase of the study
laboratory or diagnostic test abnormalities that results in the withdrawal of the patient from the study, is associated with clinical signs and symptoms or a serious adverse event (SAE), or requires medical treatment or further diagnostic work-up, or is considered by the study Investigator to be clinically significant. NOTE: Abnormal laboratory test results at the screening visit that preclude a patient from entering the study or receiving study treatment are not considered AEs, but is evaluated to monitor data from patients who do not meet screening criteria.

### Recording and Reporting Adverse Events

For the purpose of AE recording, the AE reporting period (or study period) is defined as the time period from signature of the ICF through 30 days following the last administration of study drug (or chemotherapy).

The clinical course of each AE is monitored at suitable intervals until resolved or stabilized or returned to baseline, or until the patient is referred to the care of a local health care professional, or until a determination of a cause unrelated to the study drug or study procedure is made.

The onset and end dates, duration (if the AE lasts less than 24 hours), action taken regarding study drug, treatment administered, and outcome for each AE must be recorded in the CRF. The relationship of each AE to study drug treatment and study procedures, and the severity and seriousness of each AE, as judged by the Investigator, must be recorded as described below.

### Severity of an Adverse Event

The severity of each AE must be recorded as one of the choices presented in the following scale:

The severity of each AE is graded according to the National Cancer Institute Common Terminology Criteria for Adverse Events (NCI CTCAE) current version 4.0.

AEs that are not included in the NCI CTCAE lists are graded according to the NCI CTCAE general guideline for grades as follows:
Grade 1 Mild; asymptomatic or mild symptoms; clinical or diagnostic observations only; intervention not indicated
Grade 2 Moderate; minimal, local intervention, or non-invasive intervention indicated; limiting age-appropriate instrumental activities of daily living (ADL), eg preparing meals, shopping for groceries or clothes, using the telephone, managing money
Grade 3 Severe or medically significant but not immediately life-threatening; hospitalization or prolongation of hospitalization indicated; disabling; limiting self care ADL, eg bathing, dressing and undressing, feeding self, using the toilet, taking medications, and not bedridden.
Grade 4 Life-threatening consequences; urgent intervention indicated
Grade 5 Death related to AE

Relationship of an Adverse Event to the Study Drug

The relationship of an AE to the study drug is characterized as shown below in Table 8.

TABLE 8
Characterization of Relationship Between AE and Study Drugy

| Term | Definition | Clarification |
|---|---|---|
| No | This category applies to those | An adverse experience may be considered no |
| reasonable | AEs which, after careful | reasonable possibility if it is clearly due to |
| possibility | consideration, are clearly due | extraneous causes or when (at least 2 of the |
| | to extraneous causes (disease, | following): |
| | environment, etc) or to those | it does not follow a reasonable |
| | AEs, which, after careful | temporal sequence from the |
| | medical consideration at the | administration of the test drug. |
| | time they are evaluated, are | it could readily have been produced by |
| | judged to be unrelated to the | the patient's clinical state, |
| | study drug. | environmental or toxic factors, or other |
| | | modes of therapy administered to the |
| | | patient. |
| | | it does not follow a known pattern of |
| | | response to the test drug. |
| | | it does not reappear or worsen when |
| | | the drug is re-administered. |
| Reasonable | This category applies to those | An adverse experience may be considered |
| possibility | AEs for which, after careful | reasonable possibility related if or when (at |
| | medical consideration at the | least 2 of the following): |
| | time they are evaluated, a | it follows a reasonable temporal |
| | connection with the test drug | sequence from administration of the |
| | administration cannot be | drug. |
| | ruled out with certainty or felt | it could not be reasonably explained |
| | with a high degree of | by the known characteristics of the |
| | certainty to be related to the | patient's clinical state, environmental |
| | study drug. | or toxic factors, or other modes of |
| | | therapy administered to the patient. |
| | | it disappears or decreases on cessation |
| | | or reduction in dose. There are |
| | | important exceptions when an AE |
| | | does not disappear upon |
| | | discontinuation of the drug, yet drug- |
| | | relatedness clearly exists. |
| | | it follows a known pattern of response |
| | | to the test drug. |

### Withdrawal Due to an Adverse Event

Any patient who experiences an AE may be withdrawn from the study at any time at the discretion of the Investigator. If a patient is withdrawn wholly or in part because of an AE, both the AEs page and termination page of the CRF will be completed at that time.

If a patient is withdrawn from the study for multiple reasons that include AEs, the termination page of the CRF should indicate that the withdrawal was related to an AE. An exception to this requirement will be the occurrence of an AE, which in the opinion of the Investigator is not severe enough to warrant discontinuation but which requires the use of a prohibited medication, thereby requiring discontinuation of the patient. In such a case, the reason for discontinuation would be needed to take a prohibited medication, not the AE.

### Clinical Laboratory Tests

All clinical laboratory test results outside of the reference range are interpreted by the Investigator using the following categories:
abnormal but not a clinically significant worsening
abnormal and a clinically significant worsening

A laboratory test result that has significantly worsened (according to medical judgment) compared with the baseline result is recorded in the CRF as an AE and monitored. An AE includes a laboratory or diagnostic test abnormality (once confirmed by repeat testing) that results in the withdrawal of the patient from the study, the temporary or permanent cessation of treatment with study drug, or requires medical treatment or further diagnostic work-up.

Clinical laboratory tests (biochemistry, hematology and optionally, urinalysis) are performed at screening, baseline, Day 1, and Day 21 of each chemotherapy cycle.

All the clinical laboratory tests are performed by a central laboratory, including ANC at screening. For medical decisions, ANC results are also analyzed at the local sites.

### Biochemistry

The following serum biochemistry tests are performed: calcium; phosphate; sodium; potassium; chloride; bicarbonate or carbon dioxide; glucose; blood urea nitrogen (BUN); creatinine; cholesterol; uric acid; Alanine aminotransferase (ALT); Aspartate aminotransferase (AST); LDH; gamma-glutamyl transpeptidase (GGT); alkaline phosphatase; creatine phosphokinase; total protein; albumin; total bilirubin; direct bilirubin; and indirect bilirubin.

### Hematology

The following hematology tests are performed: hemoglobin; hematocrit; RBC count; platelet count; ANC; and WBC count and differential count (polymorphonuclear leukocytes (neutrophils), lymphocytes, eosinophils, monocytes, basophils, and optionally atypical lymphocytes).

### Urinalysis

Urinalysis include testing for the following: protein; glucose; ketones; blood (hemoglobin); pH; specific gravity; and microscopic (bacteria, RBCs, WBCs, casts, and crystals).

### Other Clinical Laboratory Tests

Other clinical laboratory tests are performed to ensure the safety of the patients, but are not an assessment of the safety of the study drug.

### Human Chorionic Gonadotropin Tests

Urine pregnancy tests are performed for all female patients of child-bearing potential at the screening visit. Any patient becoming pregnant during the study is withdrawn. All pregnancies that occur during the study, or within 14 days of completion of the study, are to be reported immediately to the individual identified in the clinical study personnel contact information section of this protocol, and the Investigator must provide the Sponsor, by facsimile, with a signed pregnancy tracking form. All patients who become pregnant are monitored to the completion or termination of the pregnancy. If the pregnancy continues to term, the outcome (health of the infant up to 8 weeks of age) is reported to the Sponsor. Any complications will be reported as an AE or SAE, as appropriate.

### Spleen Sonography

A sonographic examination of the spleen is performed at baseline, at the end of cycle evaluation, and at the end of study visit.

### Vital Signs

In accordance with the schedule of study procedures shown in Tables 5 and 6, vital signs including systolic and diastolic blood pressure, pulse rate, and respiration rate. Vital signs are recorded at screening, baseline, Day 1, and at the end of cycle (Day 21).

### Temperature

Temperature will be measured at screening, baseline, on Days 1, 2, 3, 5, 7, 9, 11, 13, 15, 21, and EOV.

### Electrocardiography

In accordance with the schedule of study procedures shown in Tables 5 and 6 standard triplicate 12-lead ECGs are performed. Three sets of triplicates are taken at the screening visit (triplicates one minute apart, and each set 5 minutes apart). At all other visits, 1 set of triplicates are taken 1 minute apart. ECG should be performed following the patient being in a supine position for 5 minutes. ECGs will be transferred electronically to a central reader who interprets the reading.

The QT/QTc interval is determined from at least 3 to 5 cardiac cycles (heart beats).

### Physical Examinations

In accordance with the schedule of study procedures in Tables 5 and 6 physical examination is performed and documented by the Investigator or a qualified designee at screening, baseline (abbreviated exam only), Day 1, and Day 21. The examination is based on the following body systems: eyes, ears, nose, throat, respiratory system, cardiovascular system, abdomen, musculoskeletal system, skin, lymph nodes, and neurological system. Height and body weight are also determined.

Any abnormal findings assessed by the Investigator as clinically significant should be recorded in the relevant CRF modules (eg AEs, medical history, etc.).

### Tolerability at the Injection Site

Local tolerability at the balugrastim injection site is assessed at 1 hour (±10 min) and at 24 hours (±1 h) following balugrastim administration. The site is assessed for the presence and severity of pain, erythema/redness, ecchymosis, and induration.

Severity of any reaction should be assessed as described in Table 9. At the discretion of the Investigator, severe cases are recorded as an AE.

TABLE 9
Local Tolerability Assessment Scale

| Reaction | Severity grade | Parameter |
|---|---|---|
| Pain | 0 | Absent |
| | 1 | Painful on touch |
| | 2 | Painful when limb is moved |
| | 3 | Spontaneously painful |

| | |
|---|---|
| Erythema/Redness | Record surface diameter in mm, if ≧5 mm |
| Ecchymosis | Record surface diameter in mm, if ≧5 mm |
| Induration | Record surface diameter in mm, if ≧5 mm |

### Immunogenicity Assessments

In accordance with the schedule of study procedures in Table 5 and Table 6, blood is drawn for the assessment of antibodies against balugrastim. At follow-up visits, approximately on Days 180 and 365 after the first balugrastim administration, concomitant medication (with regard to G-CSF only) will also be documented.

Immunogenicity tests to monitor the development of ADAs to balugrastim is taken prior to balugrastim administration at Cycle 1 and Cycle 4 (pre-dose), at the EOS and during follow up visits (approximately on Days 180 and 365 [±2 weeks]) as part of the study's safety follow up measures. Each test will require a 3 mL blood sample.

All samples are collected in vacutainer tubes bearing at a minimum the patient's number, study number, study drug (optionally), dose (optionally), collection and date.

The blood samples are kept at room temperature (20-25° C.) for 60-90 minutes for clotting. Alternatively, the blood samples can be kept at 2-8° C. for 2-3 hours for clotting. Following clotting, the samples will be centrifuged at ˜1000 g for ˜10 minutes at ambient temperature. Adequate measures are taken to prevent samples from heating significantly during centrifugation.

Separated serum is transferred, in approximately equal portions, into two cryovial tubes (primary aliquot A and back-up aliquot B) and stored in an ultralow freezer at ≦65° C. until shipped to a central or bioanalytical laboratory. Storage at ≦20° C. is acceptable if a ≦65° C. freezer is not available for a period of up to 2 months. The listed temperatures must be maintained. Sample labels should include patient number, study number, study drug (optionally), dose (optionally), collection date and indication that it is an ADA sample and aliquot (A or B).

The actual times and dates of sampling are recorded on the CRF.

Balugrastim ADA is analyzed at Teva Biopharmaceuticals USA (9410 Key West Avenue, Rockville Md., 20850 USA) using validated ELISA methods for detection of binding ADAs.

Additional details for sample collection, processing, and shipment are included in the separate immunogenicity laboratory guidelines.

At the follow-up visits, any use of filgrastim, pegfilgrastim, lenograstim, biosimilars or other investigational WBC growth factors since the EOS assessment will be documented.

### Survival and Tumor Progression

Survival is documented at the follow-up visits on Day 180 and Day 365.

### Total Blood Volume

Blood sampling is performed using an indwelling catheter instead of multiple venipunctures to minimize patient's discomfort. In addition, the frequency and volume of blood samples have been set to the minimum that allows the study objectives to be achieved.

The same volume of blood is drawn from male and female patients irrespective of their age.

According to the EU guidelines, a maximum of 36 mL total amount of blood drawn within 4 weeks is the acceptable limit in a child weighing 15 kg. Therefore, the blood volumes for the youngest cohort are planned to be below 36 mL total limit for the combined screening and treatment periods. If a patient in the oldest cohort weighs less than 16 kg, the number of PK assessments for that patient should be adjusted to those of the youngest cohort. The blood volumes drawn for patients weighing 10 to kg are also in accordance with the European Medicines Agency's recommendations (24 mL).

### Assessment of Pharmacokinetics/Pharmacodynamics

### Pharmacokinetic Assessment

PK parameters are calculated for each subject using non-compartmental analysis of serum balugrastim concentration-time data.

The planned PK parameters to be calculated are:

Area under the curve to the time of last measurable concentration (AUC0-tlast).

Area under the curve from O-time extrapolated to infinity (AUC0-inf).

Maximum observed serum concentration (Cmax).

Time to maximum observed serum concentration (Tmax).

Slope of terminal phase (λz).

Terminal phase half-life (T½).

Mean residence time (MRT).

Apparent systemic clearance (CL/F).

Apparent volume of distribution, terminal phase (Vz/F).

Pharmacokinetic Blood Sampling and Variables

Blood samples for PK analysis of 0.5 mL are sequentially collected using an indwelling catheter. All samples are collected in vacutainer tubes bearing at a minimum the patient's number, study number, study drug (optionally), dose (optionally), nominal collection date and time (study hour).

The actual time of blood sampling are recorded directly in the source documentation and then transferred into the CRF.

Details for sample collection and processing are included in the separate PK laboratory guidelines.

Table 10 summarizes the allowed limits for deviations from PK sampling times. The actual time of sample collection will be documented in the CRF.

TABLE 10
Allowed Limits for Deviations from Pharmacokinetic
Sampling Times

| Scheduled sampling | |
|---|---|
| time | Allowed Deviation |
| Prior to balugrastim | 0 to 60 min prior to |
| dose | balugrastim dose |
| 6 h post dose | ±10 min |
| 12 h post dose | ±1 hour |
| 24 h (1 day) post dose | ±1 hour |
| 72 h (3 days) post dose | ±2 hours |
| 120 h (5 days) post dose | ±2 hours |
| 168 h (7 days) post dose | ±2 hours |
| 216 h (9 days) post dose | ±2 hours |

Shipment of Pharmacokinetic Samples

Information regarding sample shipment is provided in the separate PK laboratory guidelines.

Pharmacokinetic Assay Methodology

Balugrastim serum concentrations is analyzed at Teva Biopharmaceuticals USA (9410 Key West Avenue, Rockville Md., 20850 USA) using a validated ELISA method.

Pharmacodynamic Assessments

Primary Pharmacodynamic Variable

The primary PD variable for this study is:

Area under the curve of ANC (AUCANC)

Secondary Pharmacodynamic parameters

ANC nadir (measured in days), which is the lowest ANC recorded

Time to ANC nadir, which is the time from the beginning of chemotherapy up to the occurrence of the ANC nadir

Time to ANC recovery (ANC>1.5×109/L) from nadir in all treatment cycles

Pharmacodynamic Sample Schedule

A total of 9 blood samples are collected for PD assessments. The actual time of sample collection will be documented in the source records and in the CRF.

Pharmacodynamic Sample Collection and Processing

PD assessments of ANC are require 1.0 mL blood at each time point.

All samples are collected in ethylenediaminetetraacetic acid (EDTA) microtainer tubes bearing at a minimum, the patient's number, study number, collection date and time (hour).

All PD samples for ANC are sent to the central laboratory for evaluation. Details of blood sample handling, storage and shipment are described in a study specific clinical laboratory manual.

ANC is determined using a standardized method at the central laboratory.

Total Blood Volume

Blood sampling is performed using an indwelling catheter instead of multiple venipunctures to minimize patient's discomfort. In addition, the frequency and volume of blood samples have been set to the minimum that will allow the study objectives to be achieved.

The same volume of blood is drawn from male and female patients irrespective of their age.

According to the EU guidelines (EU: Ethical considerations for clinical trials, 2008), a maximum of 36 mL total amount of blood drawn within 4 weeks is the acceptable limit in a child weighing 15 kg. If a patient in the oldest cohort weighs less than 16 kg, the number of PK assessments for that patient should be adjusted to those of the youngest cohort. The blood volumes drawn for patients weighing 10 to 15 kg are also in accordance with the European Medicines Agency's recommendations (24 mL).

The total blood volumes required for the study assessments are described in Table 11. Please refer to Section O, Section O, Section O, and Section 0 describing the study assessments for individual sample volumes for PK, PD, clinical laboratory, and immunogenicity evaluations, respectively

TABLE 11
Total Blood Volume

| | | | | | | Day 24 | | |
|---|---|---|---|---|---|---|---|---|
| | Pre-study | C1 | C2 | C3 | C4 | (EOT) | EOS | Post-study |
| Safety | 2 × 2 = 4 mL | — | — | — | — | 2 × 1 = | — | — |
| (2 mL) | | | | | | 2 mL | | |
| PK | — | 8 × 0.5 = | — | 8 × 0.5 = | — | — | — | — |
| (0.5 mL) | | 4 mL | | 4 mL | | | | |
| PD (1 mL) | 1 × 1 = 1 mL | 12 × 1 = | 8 × 1 = | 12 × 1 = | 8 × 1 = | — | 1 × 1 = | — |
| | | 12 mL | 8 mL | 12 mL | 8 mL | | 1 mL | |
| IG (3 mL) | 3 × 1 = 3 mL | 3 × 1 = | — | — | 3 × 1 = | — | — | 3 × 3 = |
| | | 3 mL | | | 3 mL | | | 9 mL |
| Total per | 8 mL | 19 mL | 8 mL | 16 mL | 11 mL | 2 mL | 1 mL | 9 mL |
| cycle | | | | | | | | |

Total volume per study = 74 mL
C = cycle;
EOS = end of study visit;
EOT = end of treatment;
IG = immunogenicity;
PK = pharmacokinetics;
PD = pharmacodynamics.

C=cycle; EOS=end of study visit; EOT=end of treatment; IG=immunogenicity; PK=pharmacokinetics; PD=pharmacodynamics.

It is estimated that a total volume of 74 mL will be drawn for all assessments for the whole study including the follow up phase.

For the pre study phase and Cycle 1, a total volume of 27.0 mL will be drawn. This volume is below the acceptable limit of 36 mL in a child weighting 15 kg. The total volume drawn for children weighing 10-15 kg is within the acceptable limit of 24 mL.

### Example 2

The pharmacokinetics and efficacy of balugrastim in patients less than 18 years of age and receiving VDC/IE, VIDE, IVA, or IVAd chemotherapy are evaluated.

Balugrastim is administered in a composition comprising 20 mM sodium phosphate, 180 mM mannitol, 60 mM trehalose dehydrate, and 0.01% (w/v) polysorbate 80 at pH 6.0. Either 300 μg/kg or 670 μg/kg balugrastim are administered subcutaneously at about 24 hours after the last chemotherapy treatment (i.e., on day 3 in patients receiving VDC/IE, IVA, IVAd and on day 4 in patients receiving VIDE). Prior to receiving each cycle of therapy, patients have an absolute neutrophil count (ANC)>1.5×10⁹/L and platelets>100×10⁹/L³.

Subjects are sampled for serum balugrastim concentrations over the course of the study. The drug is detected using a sandwich enzyme-linked immunosorbent assay (ELISA) specific for balugrastim. The serum drug concentration-time data are subjected to PK analysis. The following parameters are obtained: area under the curve (AUC_{0-∞}), maximum concentration (Cmax), and half-life (_{t1/2}).

The AUC range for patients treated with 300 μg/kg balugrastim after chemotherapy is 1,000 to about 50,000 ng/mL*hr or about 5,000 to about 75,000 ng/mL*hr. The AUC for patients treated with 670 μg/kg balugrastim is about 5,000 to about 400,000 ng/mL*hr.

The Cmax for patients treated with 300 μg/kg balugrastim after chemotherapy is about 25 to about 750 ng/mL. The Cmax for patients treated with 670 μg/kg balugrastim after chemotherapy is about 40 to about 1500 ng/mL or about 40 to about 2000 ng/mL. The Cmax for balugrastim is achieved within one day of administration of balugrastim and gradually falls to undetectable levels by day 10.

After a dose of 300 ug/kg, the balugrastim elimination half-life is about 20 to 75 hours. After a dose of 670 ug/kg, the balugrastim median elimination half-life is approximately 10 to 70 hours.

Balugrastim is well tolerated and results in a dose-dependent rise in WBC and ANC. ANC and WBC return to normal levels by day 15. Lower rates of neutropenia and febrile neutropenia than expected with VDC/IE, VIDE, IVA, or IVAd therapy are observed following administration of balugrastim. Less than 10% of subjects receiving balugrastim experience febrile neutropenia. The mean duration of severe neutropenia is less than 8 days. In some patients, the mean duration of severe neutropenia after IE is less (e.g., less than 2 days) than after VDC (e.g., less than 6 days). Some younger pediatric patients experience longer durations of severe neutropenia than some older pediatric patients.

1. A method of treating or preventing neutropenia in a human subject comprising administering to a human subject exhibiting neutropenia or at risk of developing neutropenia 300 μg/kg to 670 μg/kg of recombinant human albumin-human granulocyte colony stimulating factor, wherein the human subject is less than 18 years old.

2. The method according to claim 1, wherein the subject has a non-myeloid malignancy that is a solid tumor.

3. The method according to claim 1, wherein 300 μg/kg of recombinant human albumin-human granulocyte colony stimulating factor is administered to the subject.

4. The method according to claim 1, wherein 670 μg/kg of recombinant human albumin-human granulocyte colony stimulating factor is administered to the subject.

5. The method of claim 1, wherein recombinant human albumin-human granulocyte colony stimulating factor is administered at least 18 hours after administration of a myelosuppressive anti-cancer drug.

6. The method according to claim 1, wherein the myelosuppressive anti-cancer drug comprises Vincristine, Doxorubicin, Cyclophosphamide, Ifosfamide, and Etoposide (VDC/IE).

7. The method according to claim 1, wherein the myelosuppressive anti-cancer drug comprises Vincristine, Ifosfamide, Doxorubicin, and Etoposide (VIDE).

8. The method according to claim 1, wherein the myelosuppresive anti-cancer drug comprises Ifosfamide, Vincristine, and Actinomycin D (IVA).

9. The method according to claim 1, wherein the myelosuppresive anti-cancer drug comprises Ifosfamide, Vincristine, Actinomycin D, and Doxorubicin (IVAd).

10. The method according to claim 1, wherein said recombinant human albumin-human granulocyte colony stimulating factor is administered in a composition comprising sodium phosphate, mannitol, trehalose dihydrate, and polysorbate 80.

11. A method of decreasing the incidence of infection, as manifested by febrile neutropenia, in a human subject with a non-myeloid malignancy and receiving at least one myelosuppressive anti-cancer drug associated with a clinically significant incidence of febrile neutropenia, comprising administering to the subject 300 μg/kg to 670 μg/kg recombinant human albumin-human granulocyte colony stimulating factor, wherein the human subject is less than 18 years old.

12. The method according to claim 11, wherein the human subject is 2-11 years old.

13. The method according to claim 11, wherein the human subject is 12-17 years old.

14. The method according to claim 11, wherein the non-myeloid malignancy is a solid tumor.

15. The method according to claim 11, wherein the duration of severe neutropenia is reduced.

16. The method according to claim 11, wherein administering recombinant human albumin-human granulocyte colony stimulating factor induces a rise in white blood cells (WBC).

17. The method according claim 11, wherein the number of neutrophils is increased in the subject.

18. The method according to claim 11, wherein 300 μg/kg of recombinant human albumin-human granulocyte colony stimulating factor is administered to the subject.

19. The method according to claim 11, wherein 670 μg/kg of recombinant human albumin-human granulocyte colony stimulating factor is administered to the subject.

20. The method of claim 11, wherein recombinant human albumin-human granulocyte colony stimulating factor is administered at least 18 hours after administration of the myelosuppressive anti-cancer drug.

21. The method of claim 20, wherein the recombinant human albumin-human granulocyte colony stimulating factor is administered at least 24 hours after administration of the myelosuppressive anti-cancer drug.

22. The method according to claim 11, wherein the myelosuppressive anti-cancer drug comprises Vincristine, Doxorubicin, Cyclophosphamide, Ifosfamide, and Etoposide (VDC/IE).

23. The method according to claim 11, wherein the myelosuppressive anti-cancer drug comprises Vincristine, Ifosfamide, Doxorubicin, and Etoposide (VIDE).

24. The method according to claim 11, wherein the myelosuppresive anti-cancer drug comprises Ifosfamide, Vincristine, and Actinomycin D (IVA).

25. The method according to claim 11, wherein the myelosuppresive anti-cancer drug comprises Ifosfamide, Vincristine, Actinomycin D, and Doxorubicin (IVAd).

26. The method according to claim 11, wherein absolute neutrophil count (ANC) and WBC return to normal by day 15 after administration of the anti-cancer drug.

27. The method according to claim 11, wherein said recombinant human albumin-human granulocyte colony stimulating factor is administered in a composition comprising sodium phosphate, mannitol, trehalose dihydrate, and polysorbate 80.

28. The method according to claim 27, wherein the composition is at a pH of 5.8-6.2.

29. The method according to claim 27, wherein the composition is in the form of a lyophilized cake prior to administration.
